(19) United States
(12) Patent Application Publication
(10) Pub. No.: US 2023/0243811 A1
(43) Pub. Date: Aug. 3, 2023

(54) IDENTIFICATION OF TRANSPLANTED HUMAN CELLS

(71) Applicants: UNIVERSITY OF ROCHESTER, Rochester, NY (US); UNIVERSITY OF CØPENHAGEN, Copenhagen (DK); RIGSHOPITALET (REGH), Copenhagen (DK)

(72) Inventors: Steven A. GOLDMAN, Webster, NY (US); Abdellatif BENRAISS, Astoria, NY (US); Gitte Moos KNUDSEN, Cøpenhagen (DK)

(21) Appl. No.: 17/920,140

(22) PCT Filed: Apr. 20, 2021
(86) PCT No.: PCT/US2021/028206

§ 371 Date: Oct. 20, 2022

Related U.S. Application Data
(60) Provisional application No. 63/012,709, filed on Apr. 20, 2020.

(51) Int. Cl. G01N 33/50; C12N 15/86

(52) U.S. Cl. CPC G01N 33/5088; C12N 15/86; C12N 2510/00

(57) ABSTRACT

The present disclosure is directed to systems for in vivo tracking of target cells resulting from implantation of a preparation of cells. The present disclosure is further directed to in vivo methods of tracking a preparation of cells implanted in a subject, and of preparations of cells.

This application claims the priority benefit of U.S. Provisional Patent Application Ser. No. 63/012,709, filed Apr. 20, 2020, which is hereby incorporated by reference in its entirety.

### FIELD

The present disclosure relates to systems, methods, and compositions for in vivo tracking of a preparation of cells and its progeny after the preparation of cells has been implanted into a subject.

### BACKGROUND

Cell transplantation and cell replacement therapies have emerged as promising treatments for many diseases. See, e.g., Buzhor et al., “Cell-Based Therapy Approaches: The Hope for Incurable Diseases,” Regen. Med. 9(5):649-72 (2014). For example, both myelin disorders and appropriate glial-based neurodegenerative conditions may be compelling targets for cell-based therapy. See Goldman, “Progenitor Cell-Based Treatment of Glial Disease,” Prog. Brain Res. 231:165-189 (2017) (“Goldman”). Human glial progenitor cells can generate both oligodendrocytes and astrocytes, and are thus promising reagents by which to concurrently restore myelin to injured CNS, while simultaneously addressing disorders of astrocytic function. See Goldman at 165-189.

Cell transplantation and cell replacement therapies can be complicated by uncertainty regarding the ultimate fate of the transplanted cells, as well as the risk for aberrant differentiation and tumorigenesis. For example, it has been shown that while both fetal and adult human-derived glial progenitor cells (GPCs) were able to myelinate dysmyelinated brain tissue, adult GPCs did so more rapidly and efficiently, but manifested less expansion and migratory potential in vivo. See Goldman at 165-189. In contrast, fetal GPCs emigrated more widely and engrafted more efficiently than did adult cells, and exhibited context-dependent differentiation as astrocytes or oligodendrocytes. See Goldman at 165-189.

Thus, tracking of transplanted cells in vivo to assess, e.g., cell delivery, retention, viability, and differentiation in patients will be critical for improving clinical outcomes.

The present disclosure is directed to overcoming deficiencies in the art.

### SUMMARY

The present disclosure relates to systems, methods, and compositions for in vivo tracking of a preparation of cells and its progeny after the preparation of cells has been implanted into a subject. Cells of a preparation of implantable cells are engineered to express one or more reporter molecules, i.e., cell surface binding molecules, where the gene expression pattern of the reporter molecules is characteristic of the preparation of cells, its progeny, and/or differentiated cells thereof, that are of interest to track after implantation. Expression of the one or more cell surface binding molecules is detected by binding with a labeled-binding partner. The anatomical position and identity of cells expressing the reporter molecule(s) can be generated from images of the location of the labeled binding partner in the subject's body.

Accordingly, a first aspect of the present disclosure relates to a system for in vivo tracking of target cells resulting from implantation of a preparation of cells. This system comprises one or more recombinant genetic constructs, where each construct comprises a regulatory sequence driving target cell-type specific gene expression, and a nucleotide sequence encoding a cell surface binding molecule, where the nucleotide sequence is positioned 3′ to the regulatory sequence driving cell-type specific gene expression of the recombinant genetic construct. The system further includes a preparation of cells, wherein cells of the preparation are stably transduced with the one or more recombinant genetic constructs, wherein the cell surface binding molecule encoded by each of the one or more recombinant constructs is not endogenously expressed by cells of the preparation, and whereby the regulatory sequence driving cell-type specific gene expression is activated when present in the target cell to express the cell surface binding molecule in the target cell. The system further includes one or more radiolabeled binding molecules that bind specifically to the cell surface binding molecule encoded by the one or more recombinant genetic constructs.

Another aspect of the present disclosure relates to a system for in vivo tracking of target cells resulting from implantation of a preparation of cells. This system comprises one or more recombinant genetic constructs, where each construct comprises a first nucleotide sequence of a gene expressed in a target cell-specific manner, a cell surface binding molecule encoding nucleotide sequence, where the nucleotide sequence is positioned 3′ to the first nucleotide sequence of the recombinant construct, and a second nucleotide sequence from the same gene as the first nucleotide sequence expressed in the target cell-specific manner, said second nucleotide sequence located 3′ to the nucleotide sequence encoding the cell surface binding molecule. The system further includes a preparation of cells, wherein cells of the preparation are genetically modified with the one or more recombinant genetic constructs to express the cell surface binding molecule in tandem with the gene expressed in the target cell-specific manner, wherein the cell surface binding molecule is not endogenously expressed by said target cells. The system further includes one or more radiolabeled binding molecules that bind specifically to the cell surface binding molecule encoded by the one or more recombinant genetic constructs.

Another aspect of the present disclosure relates to an in vivo method of tracking a preparation of transplanted cells in a subject. This method involves providing the system described herein where the system comprises one or more recombinant genetic constructs as described herein, a preparation of cells, wherein cells of the preparation are stably transduced with the one or more recombinant genetic constructs, and one or more radiolabeled binding molecules that bind specifically to the cell surface binding molecule encoded by the one or more recombinant genetic constructs. This method further involves implanting the preparation of cells into the subject, and administering one or more radiolabeled molecules that bind to a cell surface binding molecule encoded by the genetic construct expressed by said preparation of cells, and detecting the radiolabeled molecule bound to its cognate cell surface binding molecule expressed by implanted cells of the preparation, thereby tracking cells of the preparation in the subject.

Another aspect of the present disclosure relates to a preparation of cells, wherein the cells of the preparation are stably transduced with one or more recombinant genetic constructs, each genetic construct comprising a regulatory sequence driving target cell-type specific gene expression, and a nucleotide sequence encoding a cell surface binding molecule, wherein said nucleotide sequence is positioned 3′ to the regulatory sequence driving target cell-type specific gene expression, and wherein the cell surface binding molecule is not endogenously expressed by cells of the preparation.

Another aspect of the present disclosure relates to a preparation of cells, wherein the cells of the preparation are genetically modified with one or more recombinant genetic constructs, each construct comprising a first nucleotide sequence of a gene expressed in a target cell-specific manner, a cell surface binding molecule encoding nucleotide sequence, wherein the nucleotide sequence is positioned 3′ to the first nucleotide sequence of the recombinant genetic construct, and wherein the cell surface binding molecule is not endogenously expressed by cells of said preparation, and a second nucleotide sequence from the same gene as the first nucleotide sequence expressed in the cell-specific manner, said second nucleotide sequence located 3′ to the nucleotide sequence encoding the cell surface binding molecule.

### BRIEF DESCRIPTION OF THE DRAWINGS

FIGS. 1A-1D show Uniform Manifold Approximation and Projection (UMAP) projections of single-cell RNAseq expression levels of selected receptors in HAD100 cells colored according to cell population (FIG. 1D) and the expression of TSPO, HTR2A, and SLC6A3 (FIG. 1A), HTR4 and DRD2 (FIG. 1B), and HTR1B and SLC6A4 (FIG. 1C). Expression levels of SOX10 (FIG. 1C) and AQP4 (FIG. 1B) are for reference. Cell populations are glial progenitor cells (GPC), immature oligodendrocytes, oligodendrocytes, and astrocytes (astros). The UMAP method is described, for example, in Becht et al., “Dimensionality Reduction for Visualizing Single-Cell Data using UMAP,” Nature Biotechnology 37:38-44 (2019), which is hereby incorporated by reference in its entirety.

FIGS. 2A-2C show expression levels of selected receptors in HAD100 cells. Expression levels are expressed as transcripts per million by cell population: glial progenitor cells (GPC), immature oligodendrocytes, oligodendrocytes, astrocytes. The average expression level across population is also shown. Expression levels are shown for TSPO, HTR2A, and SLC6A3 (FIG. 2A), HTR4 and DRD2 (FIG. 2B), and HTR1B and SLC6A4 (FIG. 2C). Expression levels of SOX10 (FIG. 2C) and AQP4 (FIG. 2B) are for reference.

FIGS. 3A-3B show modifications to G protein binding sites useful for embodiments of the present application. FIG. 3A shows, in 2 dimensions, the position of the G protein binding site (G_{nq-GTP}) within the last loop and C-terminal part of receptor proteins of the present application. FIG. 3B shows the generic structure of a G protein receptor of the present application, in which the G protein binding site has been replaced by a HA tag.

FIG. 4 shows a schematic of an exemplary cell-specific recombinant construct for expressing a cell surface binding molecule of the present application. The construct generally comprises a regulatory sequence driving target cell-type specific gene expression. In this illustration, the regulatory sequence is a cell-type specific promoter (Promoter) for targeting expression of the cell surface binding molecule (A Receptor) in specific cell populations. This embodiment shows components of a lentivirus construct suitable for expressing G protein binding receptors, such as 5-HT4R, 5-HT2RA, 5-HT1BR, or D2R in target cell populations such as oligodendrocyte progenitor cells (OPCs), oligodendrocytes, and/or astrocytes. In this embodiment, the construct comprises a homology arm right (HAR) consisting of a 5′ long terminal repeat (LTR) region (5LTR), a cell-type specific promoter (Promoter), a modified receptor (Δ Receptor), a self-cleaving peptide (P2a), a Reporter, a microRNA124 target sequence (MIR124T), the woodchuck hepatitis virus posttranscriptional regulatory element (WPRE), and a homology arm left consisting of a 3′ LTR region (3LTR). As shown in this figure, cytomegalovirus (CVM) enhancer-chicken beta-actin promoter (CAG) can be used to target all cells, the Olig2 promoter can be used to target OPCs and oligodendrocytes, and the GFAP promoter can be used to target astrocytes. In this embodiment, the modified receptor is modified from the wild type by replacing the G protein binding site with a HA tag. In this embodiment, the reporter may be, for example, enhanced green fluorescent protein (EGFP) or cluster of differentiation 4 without cytoplasmic fragment (ACD4) for cell targeting and/or selection.

FIG. 5 shows a schematic of an exemplary knock-in construct for expressing a cell surface binding molecule in a cell-specific manner, where the cell surface binding molecule (one or more of them) is expressed in tandem with a gene expressed in a target cell-specific manner. In this embodiment, the construct comprises a right homology arm (HAR), i.e., a first nucleotide sequence of a gene expressed in a target cell-specific manner. An exemplary sequence is a sequence from the last exon within the coding sequence of the target cell-specific expressed gene. The construct further comprises an internal ribosome entry site (IRES), a modified receptor (e.g., signal incompetent cell surface binding molecule), a self-cleaving peptide (P2a), a reporter, a first polyadenylation sequence (PolyA), an elongation factor 1 alpha/constitutive promoter (EF1a), a puromycin N-acetyl-transferase (Puro), a second polyadenylation sequence (PolyA), and a left homology arm (HAL) (i.e., a second nucleotide sequence from the same gene as the first nucleotide sequence expressed in the target cell-specific manner). An exemplary sequence for the HAL corresponds to an untranslated region (UTR) 3′ of the target cell-type specific expressed gene. In this embodiment, the gene AAVS1 (which is a known safe harbor for hosting DNA transgenes with expected function) can be used to target all cell types, the platelet-derived growth factor receptor A (PDGFRa) or GPR17 genes can be used to target OPCs, Olig2 can be used to target OPCs and oligodendrocytes, and GFAP can be used to target astrocytes. In this embodiment, the modified receptor is modified from the wild type by replacing the G protein binding site (of a G protein binding receptor such as 5-HT4R, 5-HT2RA, 5-HT1BR, or D2R) with a HA tag. In this embodiment, the reporter may be, for example, enhanced green fluorescent protein (EGFP) or cluster of differentiation 4 without cytoplasmic fragment (ΔCD4) for cell targeting and/or selection.

FIG. 6 is a schematic of an exemplary lentivirus knock-in construct for expressing a Δ-Drd2 receptor in a cell-specific manner. In this embodiment, the lentivirus construct encodes a 5′ long terminal repeal (5LTR), a tetracycline response element (TRE), a modified receptor Δ-Drd2 (i.e., signal incompetent cell surface binding molecule), P2a (i.e., a self-cleaving peptide), enhanced green fluorescent protein (EGFP) (i.e., a reporter), a cytomegalovirus (CMV) enhancer-chicken beta-actin promoter (CAG Promoter), a tetracycline-controlled transcriptional activator (Tet-On-3G), a woodchuck hepatitis virus posttranscriptional regulatory element (WPRE), and a 3′ long terminal repeat (3LTR).

FIG. 7 is a bar graph showing the fold change (cpm) of HEK-293FT cells infected with lentivirus expressing LV-Δ-Drd2 (MOI=1) or untreated HEK-293FT cells. Data normalized to cpm of 293 cells untreated. *** p<0.001 by t Test.

FIGS. 8A-8B show the results of an experiment in which eight-week old mice received stereotaxic intra-striatal injection of lentivirus expressing LV-Δ-Drd2 (treated hemisphere) or sham (untreated hemisphere). FIG. 8A is a fluorescence microscopy image confirming transduction with the lentivirus construct. FIG. 8B is a bar graph showing the fold change (cpm) of untreated and treated mice. Scale bar: 20 μm.

### DETAILED DESCRIPTION

The present disclosure relates to systems, methods, and compositions for in vivo tracking of a preparation of cells and its progeny after the preparation of cells has been implanted into a subject. Cells of a preparation of implantable cells are engineered to express one or more reporter molecules, i.e., cell surface binding molecules, where the gene expression pattern of the reporter molecules is characteristic of the preparation of cells, its progeny, and/or differentiated cells thereof, that are of interest to track after implantation. Expression of the one or more cell surface binding molecules is detected by binding with a radiolabeled-binding partner. The anatomical position and identity of cells expressing the reporter molecule(s) can be generated from images of the location of the radiolabel binding partner in the subject's body.

Accordingly, a first aspect of the present disclosure relates to a system for in vivo tracking of target cells resulting from implantation of a preparation of cells. This system comprises one or more recombinant genetic constructs, where each construct comprises a regulatory sequence driving target cell-type specific gene expression, and a nucleotide sequence encoding a cell surface binding molecule, where the nucleotide sequence is positioned 3′ to the regulatory sequence driving target cell-type specific gene expression of the recombinant genetic construct. The system further includes a preparation of cells, wherein cells of the preparation are stably transduced with the one or more recombinant genetic constructs, wherein the cell surface binding molecule encoded by each of the one or more recombinant constructs is not endogenously expressed by cells of the preparation, and whereby the regulatory sequence driving target cell-type specific gene expression is activated when present in the target cell to express the cell surface binding molecule in the target cell. The system further includes one or more radiolabeled binding molecules that bind specifically to the cell surface binding molecule encoded by the one or more recombinant genetic constructs.

Another aspect of the present disclosure relates to a system for in vivo tracking of target cells resulting from implantation of a preparation of cells. This system comprises one or more recombinant genetic constructs, where each construct comprises a first nucleotide sequence of a gene expressed in a target cell-specific manner, a cell surface binding molecule encoding nucleotide sequence, where the nucleotide sequence is positioned 3′ to the first nucleotide sequence of the recombinant construct, and a second nucleotide sequence from the same gene as the first nucleotide sequence expressed in the target cell-specific manner, where the second nucleotide sequence is located 3′ to the nucleotide sequence encoding the cell surface binding molecule. The system further includes a preparation of cells, wherein cells of the preparation are genetically modified with the one or more recombinant genetic constructs to express the cell surface binding molecule(s) in tandem with the gene expressed in the target cell-specific manner, wherein the cell surface binding molecule is not endogenously expressed by said target cells. The system further includes one or more radiolabeled binding molecules which can be imaged with a scanner and which bind specifically to the cell surface binding molecule encoded by the one or more recombinant genetic constructs.

As used herein, the “recombinant genetic constructs” of the disclosure are nucleic acid molecules containing a combination of two or more genetic elements not naturally occurring together. Each recombinant genetic construct comprises a non-naturally occurring nucleotide sequence that can be in the form of linear DNA, circular DNA, i.e., placed within a vector (e.g., a bacterial vector, a viral vector, plasmid vector), or integrated into a genome.

As described in more detail infra, the recombinant genetic construct is introduced into the genome of cells of a preparation to be implanted into a subject to effectuate the expression of a cell surface binding molecule, i.e., the reporter molecule, for purposes of tracking the cell in its current state or in its differentiated state, as well as its progeny after implantation into a subject.

The “cell surface binding molecule” also referred to herein as the “reporter molecule”, is any cell surface expressed molecule possessing a binding domain for binding to a binding partner molecule, e.g., a ligand, a substrate, an antigen, etc. Suitable cell surface binding molecules include, without limitation, cell surface receptors (e.g., G-protein coupled receptors), glycoproteins, cell adhesion molecules, cell surface antigens, cell surface integrins, or cluster of differentiation (CD) molecules.

In some embodiments, the cell surface molecule is a modified cell surface molecule that is altered compared to a reference or wildtype form of the cell surface molecule. In some embodiments, the modified cell surface molecule contains one or more amino acid modifications, such as one or more amino acid substitutions, deletions, and/or insertions, compared to the reference cell surface molecule. In some embodiments, the modified cell surface molecule, such as a modified cell surface receptor, is modified to remove or disrupt one or more signaling and/or trafficking domains. In some cases, the modified cell surface molecule lacks a functional intracellular signaling domain or region involved in eliciting, mediating, activating, inhibiting, and/or transmitting cellular signaling and/or downstream activities or function, i.e., the modified cell surface molecule is rendered signal incompetent. In some embodiments, the modified cell surface molecule, e.g., a modified cell surface receptor, exhibits altered cellular internalization, cellular trafficking, enzymatic activity and/or ligand binding, compared to the wild-type or unmodified cell surface molecule. In some embodiments, the one or more amino acid modifications, such as one or more amino acid substitutions, deletions and/or insertions, including truncations, can be present in one or more of the intracellular (e.g., cytoplasmic) and/or extracellular portions of the cell surface molecule.

In some embodiments, the modified cell surface molecule is truncated, for example by a deletion of a contiguous sequence of C-terminal or N-terminal amino acid residues of a reference cell surface molecule, such as deletion of from or from about 10 to 800 amino acids, for example, at least or about at least 10, 20, 30, 40, 50, 60, 70, 80, 90, 100, 200, 300, 400, 500, 600, or more contiguous amino acids of the reference cell surface molecule. In some aspects, the modified cell surface molecule is truncated, such as by deletion of a contiguous amino acid residues of intracellular (e.g., cytoplasmic) portion of the protein, for example, present in the C-terminus portion or in the N-terminus portion proteins.

In some embodiments, the modified cell surface molecule is modified by removal of one or more residues of an active domain and replacement of those one or more residues with one or more inactive residues. In some embodiments, the one or more replacement residues can constitute a marker molecule itself. For example, in one embodiment, the one or more replacement residues is a hemagglutinin (HA) tag.

In some embodiments, the cell surface molecule is a G-protein coupled cell surface receptor protein. G-protein coupled cell surface receptor proteins are a family of membrane proteins characterized by a general structure of seven transmembrane helices. See Rosenbaum et al., “The Structure and Function of G-Protein-Coupled Receptors,” Nature 459:356-63 (2009). In some embodiments, the G-protein coupled cell surface receptor protein is modified by removing or replacing an intercellular fragment necessary to transmit an extracellular ligand binding event to the intercellular space, e.g., a portion of the G protein binding site of the reporter cell surface receptor is modified or deleted. These G protein binding site can be modified by one or more amino acid substitutions, insertions, or deletions. In some embodiments, the G protein binding site is modified by replacement of the binding site, or at least a portion of the binding site, with a sequence that is incapable of transmitting a signal. In some embodiments, the G-protein binding site, or a portion thereof, is replaced with a sequence encoding a tag, e.g., a hemagglutinin (HA) tag.

In some embodiments, the HA tag comprises the amino acid sequence YPYDVPDYA (SEQ ID NO:1), which can be encoded, for example, by the nucleic acid sequence 5′-TAC CCA TAC GAT GTT CCA GAT TAC GCT-3′ (SEQ ID NO:2) or the nucleic acid sequence 5′-TAT CCA TAT GAT GTT CCA GAT TAT GCT-3′ (SEQ ID NO:3).

In some embodiments, the cell surface binding molecule encoded by the recombinant genetic construct and expressed by the preparation of cells to be tracked in vivo is a cell surface G-coupled receptor protein. Suitable cell surface G-coupled receptor proteins include, without limitation acetylcholine receptors (muscarinic), adenosine receptors, adhesion class GPCRs, adrenoceptors, angiotensin receptors, apelin receptors, bile acid receptors, bombesin receptors, bradykinin receptors, calcitonin receptors, calcium-sensing receptors, cannabinoid receptors, chemerin receptors, chemokine receptors, cholecystokinin receptors, class Frizzled GPCRs, complement peptide receptors, corticotropin-releasing factor receptors, dopamine receptors, endothelin receptors, G protein-coupled estrogen receptors, formylpeptide receptors, free fatty acid receptors, GABAB receptors, galanin receptors, ghrelin receptor, glucagon receptor family, glycoprotein hormone receptors, gonadotrophin-releasing hormone receptors, GPR18, GPR55 and GPR119, histamine receptors, hydroxycarboxylic acid receptors, kisspeptin receptors, leukotriene receptors, lysophospholipid (LPA) receptors, lysophospholipid (S1P) receptors, melanin-concentrating hormone receptors, melanocortin receptors, melatonin receptors, metabotropic glutamate receptors, motilin receptors, neuromedin U receptors, neuropeptide FF/neuropeptide AF receptors, neuropeptide S receptors, neuropeptide W/neuropeptide B receptors, neuropeptide Y receptors, neurotensin receptors, opioid receptors, orexin receptors, oxoglutarate receptors, P2Y receptors, parathyroid hormone receptors, platelet-activating factor receptors, prokineticin receptors, prolactin-releasing peptide receptors, prostanoid receptors, proteinase-activated receptors, QRFP receptors, relaxin family peptide receptors, somatostatin receptors, succinate receptors, tachykinin receptors, thyrotropin-releasing hormone receptors, trace amine receptors, Urotensin receptors, vasopressin and oxytocin receptors, VIP and PACAP receptors, Class A Orphans, Class B Orphans, Class C Orphans, Opsin receptors, Taste 1 receptors, Taste 2 receptors. G-protein coupled receptors and resources for identifying them are described, for example, in Munk et al., “GPCRdb: the G Protein-Coupled Receptor Database—an Introduction,” Br. J. Pharmacol. 173(14):2195-2207 (2016), which is hereby incorporated by reference in its entirety.

In some embodiments, the cell surface-G-coupled receptor protein is a dopamine receptor or a serotonin receptor or transporter. Dopamine and serotonin receptors and transporters are described, for example, in Yamamoto et al., “Classification of Dopamine Receptor Genes in Vertebrates: Nine Subtypes in Osteichthyes,” Brain, Behay. Evol. 86:164-75 (2015); and Seeman, “Dopamine and Serotonin Receptors: Amino Acid Sequences, and Clinical Role in Neuroleptic Parkinsonism,” Jpn. J. Pharmacol. 71:187-204 (1996), which are each hereby incorporated by reference in their entirety. The concentration and distribution in the body of some dopamine or serotonin targets are known from, e.g., Beliveau et al, “High-Resolution In Vivo Atlas of Serotonin System,” J. Neurosci., 37:120-128 (2017), which is hereby incorporated by reference in its entirety. The nucleotide sequence encoding these exemplary cell surface binding molecules are known in the art and readily accessible via the National Center for Biotechnology Information database or the UniProtKB database. Exemplary dopamine and serotonin receptors and transporters are identified in Table 1 below.

TABLE 1
Exemplary Dopamine and Serotonin
Receptors and Transporters

| | | | NCBI |
|---|---|---|---|
| | | | Gene |
| Receptor | Gene | Organism | ID |
| Dopamine Receptor 1 | DRD1 | Human | 1812 |
| | | Mouse | 13488 |
| Dopamine Receptor 2 | DRD2 | Human | 1813 |
| | | Mouse | 13489 |
| Dopamine Receptor 3 | DRD3 | Human | 1814 |
| | | Mouse | 13490 |
| Dopamine Receptor 4 | DRD4 | Human | 1815 |
| | | Mouse | 13491 |
| Dopamine Receptor 5 | DRD5 | Human | 1816 |
| | | Mouse | 13492 |
| 5-hydroxytryptamine | HTR1A | Human | 3350 |
| (serotonin) receptor 1A | | Mouse | 15550 |
| 5-hydroxytryptamine | HTR1B | Human | 3351 |
| (serotonin) receptor 1B | | Mouse | 16150 |
| 5-hydroxytryptamine | HTR1D | Human | 3352 |
| (serotonin) receptor 1D | | Mouse | 15552 |
| 5-hydroxytryptamine | HTR1E | Human | 3354 |
| (serotonin) receptor 1E | | | |
| 5-hydroxytryptamine | HTR1F | Human | 3355 |
| (serotonin) receptor 1F | | Mouse | 15557 |
| 5-hydroxytryptamine | HTR2A | Human | 3356 |
| (serotonin) receptor 2A | | Mouse | 16180 |
| 5-hydroxytryptamine | HTR2B | Human | 3357 |
| (serotonin) receptor 2B | | Mouse | 15559 |
| 5-hydroxytryptamine | HTR2C | Human | 3358 |
| (serotonin) receptor 2C | | Mouse | 15560 |
| 5-hydroxytryptamine | HTR3A | Human | 3359 |
| (serotonin) receptor 3A | | Mouse | 15561 |
| 5-hydroxytryptamine | HTR3B | Human | 9177 |
| (serotonin) receptor 3B | | Mouse | 57014 |
| 5-hydroxytryptamine | HTR3C | Human | 170572 |
| (serotonin) receptor 3C | | | |
| 5-hydroxytryptamine | HTR3D | Human | 200909 |
| (serotonin) receptor 3D | | | |
| 5-hydroxytryptamine | HTR3E | Human | 285242 |
| (serotonin) receptor 3E | | | |
| 5-hydroxytryptamine | HTR4 | Human | 3360 |
| (serotonin) receptor 4 | | Mouse | 15562 |
| 5-hydroxytryptamine | HTR5A | Human | 3361 |
| (serotonin) receptor 5A | | Mouse | 15563 |
| 5-hydroxytryptamine | HTR6 | Human | 3362 |
| (serotonin) receptor 6 | | Mouse | 15565 |
| 5-hydroxytryptamine | HTR7 | Human | 3363 |
| (serotonin) receptor 7 | | Mouse | 15566 |

In some embodiments, the cell surface G-coupled receptor protein is a dopamine receptor (e.g., dopamine receptor 2 encoded by DRD2), a serotonin receptor, e.g., a serotonin receptor 4 (encoded by HTR4), a serotonin receptor 2 (encoded by HTR2A), or a serotonin receptor 1B (encoded by HTR1B).

In some embodiments, the cell surface-G-coupled receptor protein is the dopamine receptor 2 encoded by DRD2, the sequence of which (SEQ ID NO:4) is as follows:

MDPLNLSWYDDDLERQNWSRPFNGSDGKADRPHYNYYATLLTLLIAVIV
FGNVLVCMAVSREKALQTTTNYLIVSLAVADLLVATLVMPWVVYLEVVG
EWKFSRIHCDIFVTLDVMMCTASILNLCAISIDRYTAVAMPMLYNTRYS
SKRRVTVMISIVWVLSFTISCPLLFGLNNADQNECIIANPAFVVYSSIV
SFYVPFIVTLLVYIKIYIVLRRRRKRVNTKRSSRAFRAHLRAPLK [symbol] NCT
HPEDMKLCTVIMKSNGSFPVNRRRVEAARRAQELEMEMLSSTSPPERTR
YSPIPPSHHQLTLPDPSHHGLHSTPDSPAKPEKNGHAKDHPKIAKIFEI
QTMPNGKTRTSLKTMSRRKLSQQKEKKATQMLAIVLGVFIICWLPFFIT
HILNIHCDCNIPPVLYSAFTWLGYVNSAVNPHIYTTFNIEFRKAFLKIL
HC.

The predicted intracellular loop linking transmembrane domains 5 and 6 is shown in underline. See UnProtKB entry P14416 (DRD_HUMA). In some embodiments, the G-protein receptor binding domain is located in the intracellular loop linking transmembrane domains 5 and 6, and is altered to prevent G-protein mediated signal transduction.

In some embodiments, the cell surface-G-coupled receptor protein is the serotonin receptor 4 encoded by HTR4, the sequence of which (SEQ ID NO:5) is as follows:

MDKLDANVSSEEGFGSVEKVVLLTFLSTVILMAILGNLLVMVAVCWDRQ
LRKIKTNYFIVSLAFADLLVSVLVMPFGAIELVQDIWIYGEVFCLVRTS
LDVLLTTASIFHLCCISLDRYYAICCQPLVYRNKMTPLRIALMLGGCWV
IPTFISFLPIMQGWNNIGIIDLIEKRKFNQNSNSTYCVFMVNKPYAITC
SVVAFYIPFLLMVLAYYRIYVTAKEHAHQIQMLQRAGASSESRPQSADQ
HSTHRMRTETKAAKTLCIIMGCFCLCWAPFFVTNIVDPFIDYTVPGQVW
TAFLWLGYINSGLNPFLYAFLNKSFRRAFLIILCCDDERYRRPSILGQT
VPCSTTTINGSTHVLRDAVECGGQWESQCHPPATSPLVAAQPSDT.

The predicted intracellular loop linking transmembrane domains 5 and 6 is shown in underline. See UnProtKB entry Q13639 (5HT4R HUMAN); see also Padayatti et al., “A Hybrid Structural Approach to Analyze Ligand Binding by the Serotonin Type 4 Receptor (5-HT4),” Molecular & Cellular Proteomics 12(5):1259-71 (2013), which is hereby incorporated by reference in its entirety. In some embodiments, the G-protein receptor binding domain is located in the intracellular loop linking transmembrane domains 5 and 6, and is altered to prevent G-protein mediated signal transduction.

In some embodiments, the cell surface-G-coupled receptor protein is the serotonin receptor 2a encoded by HTR2A, the sequence of which (SEQ ID NO:6) is as follows:

MDILCEENTSLSSTTNSLMQLNDDTRLYSNDFNSGEANTSDAFNWTVDS
ENRTNLSCEGCLSPSCLSLLHLQEKNWSALLTAVVIILTIAGNILVIMA
VSLEKKLQNATNYFLMSLAIADMLLGFLVMPVSMLTILYGYRWPLPSKL
CAVWIYLDVLFSTASIMHLCAISLDRYVAIQNPIHHSRFNSRTKAFLKI
IAVWTISVGISMPIPVFGLQDDSKVFKEGSCLLADDNFVLIGSFVSFFI
PLTIMVITYFLTIKSLQKEATLCVSDLGTRAKLASFSFLPQSSLSSEKL
FQRSIHREPGSYTGRRTMQSISNEQKACKVLGIVFFLFVVMWCPFFITN
IMAVICKESCNEDVIGALLNVFVWIGYLSSAVNPLVYTLFNKTYRSAFS
RYIQCQYKENKKPLQLILVNTIPALAYKSSQLQMGQKKNSKQDAKTTDN
DCSMVALGKQHSEEASKDNSDGVNEKVSCV.

The predicted intracellular loop linking transmembrane domains 5 and 6 is shown in underline. See UnProtKB entry P28223 (5HT2A HUMAN). In some embodiments, the G-protein receptor binding domain is located in the intracellular loop linking transmembrane domains 5 and 6, and is altered to prevent G-protein mediated signal transduction.

In some embodiments, the cell surface-G-coupled receptor protein is the serotonin receptor 1b encoded by HTR1B, the sequence of which (SEQ ID NO:7) is as follows:

MEEPGAQCAPPPPAGSETWVPQANLSSAPSQNCSAKDYIYQDSISLPWK
VLLVMLLALITLATTLSNAFVIATVYRTRKLHTPANYLIASLAVTDLLV
SILVMPISTMYTVTGRWTLGQVVCDFWLSSDITCCTASILHLCVIALDR
YWAITDAVEYSAKRTPKRAAVMIALVWVFSISISLPPFFWRQAKAEEEV
SECVVNTDHILYTVYSTVGAFYFPTLLLIALYGRIYVEARSRILKQTPN
RTGKRLTRAQLITDSPGSTSSVTSINSRVPDVPSESGSPVYVNQVKVRV
SDALLEKKKLMAARERKATKTLGIILGAFIVCWLPFFIISLVMPICKDA
CWFHLAIFDFFTWLGYLNSLINPIIYTMSNEDFKQAFHKLIRFKCTS.

The predicted intracellular loop linking transmembrane domains 5 and 6 is shown in underline. See UnProtKB entry P28222 (5HT1B HUMAN). In some embodiments, the G-protein receptor binding domain is located in the intracellular loop linking transmembrane domains 5 and 6, and is altered to prevent G-protein mediated signal transduction.

In some embodiments, the cell surface binding molecule is a dopamine transporter or serotonin transporter. The nucleotide sequence encoding these exemplary cell surface binding molecules are known in the art and readily accessible via the National Center for Biotechnology Information database or the UniProtKB database. Exemplary dopamine and serotonin receptors and transporters are identified in Table 2 below.

TABLE 2
Exemplary Dopamine and Serotonin Receptors and Transporters

| | | | |
|---|---|---|---|
| Dopamine Transporter | SLC6A3 | Human | 6531 |
| | | Mouse | 13162 |
| Serotonin Transporter | SLCA4 | Human | 6532 |
| | | Mouse | 15567 |

In some embodiments, the cell surface binding molecule is the dopamine transporter encoded by SLC6A3, the sequence of which (SEQ ID NO:8) is as follows:

MSKSKCSVGLMSSVVAPAKEPNAVGPKEVELILVKEQNGVQLTSSTLTN
PRQSPVEAQDRETWGKKIDFLLSVIGFAVDLANVWRFPYLCYKNGGGAF
LVPYLLFMVIAGMPLFYMELALGQFNREGAAGVWKICPILKGVGFTVIL
ISLYVGFFYNVIIAWALHYLFSSFTTELPWIHCNNSWNSPNCSDAHPGD
SSGDSSGLNDTFGTTPAAEYFERGVLHLHQSHGIDDLGPPRWQLTACLV
LVIVLLYFSLWKGVKTSGKVVWITATMPYVVLTALLLRGVTLPGAIDGI
RAYLSVDFYRLCEASVWIDAATQVCFSLGVGFGVLIAFSSYNKFTNNCY
RDAIVTTSINSLTSFSSGFVVFSFLGYMAQKHSVPIGDVAKDGPGLIFI
IYPEAIATLPLSSAWAVVFFIMLLTLGIDSAMGGMESVITGLIDEFQLL
HRHRELFTLFIVLATFLLSLFCVTNGGIYVFTLLDHFAAGTSILFGVLI
EAIGVAWFYGVGQFSDDIQQMTGQRPSLYWRLCWKLVSPCFLLFVVVVS
IVTFRPPHYGAYIFPDWANALGWVIATSSMAMVPIYAAYKFCSLPGSFR
EKLAYAIAPEKDRELVDRGEVRQFTLRHWLKV.

The predicted N′ and C′ terminal domains are shown in underline. In embodiments, the N′ and/or C′ terminal domains are involved in signaling. See UnProtKB entry Q01959 (SC6A3_HUMAN). In some embodiments, the transporter is modified to prevent signal transduction. In some embodiments, the transporter is modified by removal or replacement of the N′ terminal portion and/or the C′ terminal portion.

In some embodiments, the cell surface binding molecule is the serotonin transporter encoded by SLC6A4, the sequence of which (SEQ ID NO:9) is as follows:

METTPLNSQKQLSACEDGEDCQENGVLQKVVPTPGDKVESGQISNGYSA
VPSPGAGDDTRHSIPATTTTLVAELHQGERETWGKKVDFLLSVIGYAVD
LGNVWRFPYICYQNGGGAFLLPYTIMAIFGGIPLFYMELALGQYHRNGC
ISIWRKICPIFKGIGYAICIIAFYIASYYNTIMAWALYYLISSFTDQLP
WTSCKNSWNTGNCTNYFSEDNITWTLHSTSPAEEFYTRHVLQIHRSKGL
QDLGGISWQLALCIMLIFTVIYFSIWKGVKTSGKVVWVTATFPYIILSV
LLVRGATLPGAWRGVLFYLKPNWQKLLETGVWIDAAAQIFFSLGPGFGV
LLAFASYNKFNNNCYQDALVTSVVNCMTSFVSGFVIFTVLGYMAEMRNE
DVSEVAKDAGPSLLFITYAEAIANMPASTFFAIIFFLMLITLGLDSTFA
GLEGVITAVLDEFPHVWAKRRERFVLAVVITCFFGSLVTLTFGGAYVVK
LLEEYATGPAVLTVALIEAVAVSWFYGITQFCRDVKEMLGFSPGWFWRI
CWVAISPLFLLFIICSFLMSPPQLRLFQYNYPYWSIILGYCIGTSSFIC
IPTYIAYRLIITPGTFKERIKSITPETPTEIPCGDIRLNAV.

The predicted N′ and C′ terminal domains are shown in underline. In embodiments, the N′ and/or C′ terminal domains are involved in signaling. See UnProtKB entry P31645 (SC6A4_HUMAN). In some embodiments, the transporter is modified to prevent signal transduction. In some embodiments, the transporter is modified by removal or replacement of the N′ terminal portion and/or the C′ terminal portion.

In embodiments, the sequence of the cell surface binding molecule according to these or any other embodiments described herein comprise one or more (e.g., 1, 2, 3, 4, 5 or more) amino acid insertions, deletions, modifications (e.g. substitution of one amino acid for another) compared to any one of SEQ ID NOs:4-9, or are otherwise substantially identical (e.g. having a sequence of at least 80%, 81%, 82%, 83%, 84%, 85%, 86%, 87%, 88%, 89%, 90%, 91%, 92%, 93%, 94%, 95%, 96%, 97%, 98%, 99% or more identical) with the entire sequence of SEQ ID NOs:4-9. It is contemplated that such variations retain the cell surface binding function of, for example, any one of SEQ ID NOs:4-9. For example, polypeptides or proteins comprising an amino acid sequence having one or more (e.g. 1, 2, 3, 4, 5, or more) conservative amino acid substitutions relative to any one of SEQ ID NOs:4-9, but retaining the cell surface binding function of the any one of SEQ ID NOs:4-9 are encompassed. Nucleic acid molecules encoding such variants are also contemplated.

Other exemplary surface receptor molecules suitable for inclusion in the recombinant genetic construct described herein include, without limitation, EpCAM, VEGFR, integrin (e.g., integrins ανβ3, α4, α4β7, α5β1, ανβ3, αν), a member of the TNF receptor superfamily (e.g., TRAIL-R1, TRAIL-R2), a member of the epidermal growth factor receptor family, PDGF Receptor, interferon receptor, folate receptor, GPNMB, ICAM-1, HLA-DR, CEA, CA-125, MUC1, TAG-72, IL-6 receptor, 5T4, GD2, GD3, prostate-specific membrane antigen (PSMA), or clusters of differentiation (e.g., CD2, CD3, CD4, CD5, CD11, CDlla/LFA-1, CD15, CD18/ITGB2, CD19, CD20, CD22, CD23/IgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5 and CD319/SLAMF7. The nucleotide sequence encoding these exemplary cell surface binding molecules are known in the art and readily accessible via the National Center for Biotechnology Information database or the UniProtKB database.

Exemplary cell surface molecules also include, without limitation, epidermal growth factor receptor (EGFR), an erbB-2 receptor tyrosine-protein kinase (errb2, HER2), an erbB-3 receptor tyrosine-protein kinase, an erbB-4 receptor tyrosine-protein kinase, a hepatocyte growth factor receptor (HGFR/c-MET) or an insulin-like growth factor receptor-1 (IGF-1 R). The nucleotide sequence encoding these exemplary cell surface binding molecules are known in the art and readily accessible to via the National Center for Biotechnology Information database or the UniProtKB database.

In some embodiments, the recombinant genetic construct of the system encodes a cell surface binding molecule containing an extracellular domain or regions containing one or more epitope(s) recognized by a radiolabeled antibody or an antigen-binding fragment thereof. The antibody or antigen-binding fragment can include polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, variable heavy chain (VH) regions capable of specifically binding the antigen, single chain antibody fragments, including single chain variable fragments (scFv), and single domain antibodies (e.g., sdAb, sdFv, nanobody) fragments. Antibodies or antigen-binding fragment thereof can include intact or full-length antibodies, including antibodies of any class or sub-class, including IgG and subclasses thereof, IgM, IgE, IgA, and IgD, or portion or fragments of a full-length antibody. In some aspects, the one or more epitopes can contain contiguous or non-contiguous sequences of a molecule or protein. In some aspects, the one or more epitope(s) is present in the extracellular portion or region of the reference cell surface molecule, such that the reference cell surface molecule can be recognized, identified or detected by the antibody or antigen-binding fragment.

Suitable recombinant genetic constructs of the system encode cell surface binding molecules that are not endogenously expressed in the cell preparation of the system that are to be tracked upon implantation. This allows for tracking and distinguishing implanted cells from cells of the same type that are endogenously present in the subject receiving the implanted cells.

As noted above, in some embodiments, the cell surface binding molecule or modified version thereof is placed under the control of a regulatory sequence driving target cell-type specific gene expression. In some embodiments, the regulatory sequence driving target cell-type specific gene expression is a gene promoter sequence of a gene that is expressed in a target cell-specific manner. In some embodiments, the regulatory sequence driving target cell-type specific gene expression is one or more cell-specific enhancer sequences that drive gene expression in a cell-type specific manner (see, e.g., Anderson et al., “An Atlas of Active Enhancers Across Human Cell Types and Tissues,” Nature 507(7493): 455-461 (2014), which is hereby incorporated by reference in its entirety). In some embodiments, the regulatory sequence driving cell-type specific gene expression is a combination of a gene promoter sequence and a cell-specific enhancer sequence.

In some embodiments, the cell surface binding molecule or modified version thereof is coupled to the expression of a gene that is selectively expressed in the target cell being tracked. Coupling the expression of the cell surface binding molecule to a gene selectively expressed in the target cell of interest can be achieved using a “knock-in” recombinant genetic construct that is designed to include a first nucleotide sequence of the gene selectively expressed in a target cell-specific manner, a cell surface binding molecule encoding nucleotide sequence positioned 3′ to the first nucleotide sequences, and a second nucleotide sequence from the same gene as the first nucleotide sequence that is selectively expressed in the target cell-specific manner, where the second nucleotide sequence is located 3′ to the cell surface binding molecule encoding nucleotide sequence. The cell surface binding molecule encoding nucleotide sequence is introduced into the genome of the cell preparation to be transplanted by, e.g., homologous recombination, and is subsequently expressed in tandem with the gene selectively expressed in the target cell of interest. Suitable genes that are expressed in a cell-specific manner and their cognate promoter sequences are known in the art and described below.

In some embodiments, the regulatory sequence driving cell-type specific gene expression or the first and second nucleotide sequences of the gene selectively expressed in a target cell-specific manner are derived from a gene that is restrictively expressed in one or more differentiated cell types. In some embodiments, the differentiated cell type is a differentiated cell type of the central nervous system. For example, in some embodiments, the differentiated cell is an oligodendrocyte. In this embodiment, the regulatory sequence driving target cell-type specific gene expression or the first and second nucleotide sequences of a cell-type specific expressed gene are derived from a gene selected from the transcription regulator SRY-box 10 (SOX10), the membrane-associated transcription factor, Myelin Regulatory Factor (MYRF), Myelin-associated Glycoprotein (MAG), or Myelin Basic Protein (MBP). In another embodiment, the differentiated cell is an astrocyte, and the regulatory sequence driving cell-type specific gene expression or the first and second nucleotide sequences of a cell-type specific expressed gene are derived from glial fibrillary acidic protein (GFAP) and/or aquaporin-4 (AQP4), which are selectively expressed in astrocytes.

In another embodiment, the differentiated cell is a neuron, and the regulatory sequence driving cell-type specific gene expression or the first and second nucleotide sequences of the cell-specific expressed gene are derived from a gene selected from synapsin 1 (SYN1), microtubule associated protein 2 (MAP2), and ELAV like RNA binding protein 4 (ELAV4). In some embodiments, the differentiated cell is a dopaminergic neuron and the regulatory sequence or first and second gene specific nucleotide sequences are derived from the tyrosine hydroxylase gene (TH) or the DOPA decarboxylase gene (DDC). In some embodiments, the differentiated cells are medium spiny neurons and cortical interneurons, and the regulatory sequence or first and second gene specific nucleotide sequences are derived the gene encoding glutamate decarboxylase 2 (GAD2, also known as GAD65) or the gene encoding glutamate decarboxylase 1 (GAD1, also known as GAD67). In some embodiments, the differentiated cell is a cholinergic neuron and the regulatory sequence or first and second gene specific nucleotide sequences are derived from CHAT.

In some embodiments, the regulatory sequence driving cell-type specific gene expression or the first and second nucleotide sequences of the gene selectively expressed in a target cell-specific manner are derived from a gene that is restrictively expressed in a progenitor cell type. In some embodiments, the progenitor cell is a glial progenitor cell and the regulatory sequence or first and second gene specific nucleotide sequences are derived from a gene selected from platelet derived growth factor receptor α (PDGFRα), CD44, GPR17, or oligodendrocyte transcription factor 2 (OLIG2).

The regulatory sequences driving cell-type specific gene expression and nucleotide sequences of the genes expressed in a target cell-specific manner in various organisms, for example mice and human, are known in the art and can be readily accessed by one of ordinary skill in the art using, e.g., the NCBI Gene ID. Exemplary progenitor and differentiated CNS target cells and target cell-specific genes from which the regulatory sequences driving cell-type specific expression and/or nucleotide sequences can be derived from are identified in Table 3 below.

TABLE 3
Exemplary CNS Cells and Cell-specific Gene Targets

| | Cell-specific | | NCBI |
|---|---|---|---|
| | Gene | | Gene |
| Cell Type | Target | Organism | ID: |
| Oligodendrocyte | SOX10 | Human | 6663 |
| | | Mouse | 20665 |
| | MYRF | Human | 745 |
| | | Mouse | 225908 |
| | MAG | Human | 4099 |
| | | Mouse | 17136 |
| | MBP | Human | 4155 |
| | | Mouse | 17196 |
| Astrocyte | GFAP | Human | 2670 |
| | | Mouse | 14580 |
| | AQP4 | Human | 361 |
| | | Mouse | 11829 |
| Neurons | SYN1 | Human | 6853 |
| | | Mouse | 20964 |
| | MAP2 | Human | 4133 |
| | | Mouse | 17756 |
| | ELAV4 | Human | 1996 |
| | | Mouse | 15572 |
| Dopaminergic | TH (tyrosine | Human | 7054 |
| Neurons | hydroxylase) | Mouse | 21823 |
| | DDC (DOPA | Human | 1644 |
| | decarboxylase) | Mouse | 13195 |
| Cholinergic | CHAT (Choline O- | Human | 1103 |
| Neurons | acetyltransferase) | Mouse | 12647 |
| Medium spiny | GAD65 | Human | 2572 |
| neurons/interneurons | | Mouse | 14417 |
| | GAD67 | Human | 2571 |
| | | Mouse | 14415 |
| Glutaminergic | SLC17A6 | Human | 57084 |
| Neurons | | Mouse | 140919 |
| | SLC17A7 | Human | 57030 |
| | | Mouse | 72961 |
| Glial Progenitor | PDGFRa | Human | 5156 |
| Cell | | Mouse | 18595 |
| | CD44 | Human | 960 |
| | | Mouse | 12505 |
| | OLIG2 | Human | 10215 |
| | | Mouse | 50913 |
| | GPR17 | Human | 2840 |
| | | Mouse | 574402 |
| Neural Progenitor | NES | Human | 10763 |
| Cells | | Mouse | 18008 |
| | SOX2 | Human | 6657 |
| | | Mouse | 20674 |
| Neuronal Progenitor | MAP2 | Human | 4133 |
| Cells | | Mouse | 17756 |
| | βIII Tubulin | Human | 10381 |
| | | Mouse | 22152 |

In another embodiment, regulatory sequences driving cell-type specific gene expression and nucleotide sequences of genes expressed in a target cell-specific manner are derived from a gene that is expressed in a differentiated cell outside of the central nervous system (CNS). Exemplary differentiated non-CNS target cells include, without limitation, adipocytes, chondrocytes, endothelial cells, epithelial cells (keratinocytes, melanocytes), bone cells (osteoblasts, osteoclasts), liver cells (cholangiocytes, hepatocytes), muscle cells (cardiomyocytes, skeletal muscle cells, smooth muscle cells), retinal cells (ganglion cells, muller cells, photoreceptor cells), retinal pigment epithelial cells, renal cells (podocytes, proximal tubule cells, collecting duct cells, distal tubule cells), adrenal cells (cortical adrenal cells, medullary adrenal cells), pancreatic cells (alpha cells, beta cells, delta cells, epsilon cells, pancreatic polypeptide producing cells, exocrine cells), lung cells, bone marrow cells (early B-cell development, early T-cell development, macrophages, monocytes), urothelial cells, fibroblasts, parathyroid cells, thyroid cells, hypothalamic cells, pituitary cells, salivary gland cells, ovarian cells, and testicular cells. Exemplary differentiated non-CNS target cells and target cell-specific genes from which the regulatory sequences driving cell-type specific gene expression and/or nucleotide sequences of the genes expressed in a target cell-specific manner can be derived from are identified in Table 4 below.

TABLE 4
Exemplary Non-CNS Cells and Cell-specific Gene Targets

| Terminally | Cell-specific | | |
|---|---|---|---|
| Differentiated | Gene | | Gene |
| Cell Type | Target | Organism | ID: |
| Adipocytes | ADIPOQ | Human | 9370 |
| | (ACRP30) | Mouse | 11450 |
| | FABP4 | Human | 2167 |
| | | Mouse | 11770 |
| | PPARG | Human | 5468 |
| | | Mouse | 19016 |
| Chondrocytes | ACAN (AGC1) | Human | 176 |
| | | Mouse | 11595 |
| | COL10A1 | Human | 1300 |
| | | Mouse | 12813 |
| | COMP | Human | 1311 |
| | | Mouse | 12845 |
| Endothelial | CDH5 | Human | 1003 |
| cells (general) | | Mouse | 12562 |
| | KDR | Human | 3791 |
| | (VEGFR3) | Mouse | 16542 |
| | PECAM1 | Human | 5175 |
| | | Mouse | 18613 |
| Endothelial | DLL4 | Human | 54567 |
| cells (arterial) | | Mouse | 54485 |
| | EFNB2 | Human | 1948 |
| | | Mouse | 13642 |
| | NRP1 | Human | 8829 |
| | | Mouse | 18186 |
| Endothelial | LYVE1 | Human | 10894 |
| cells (lymphatic) | | Mouse | 114332 |
| | PROXI | Human | 5629 |
| | | Mouse | 19130 |
| Endothelial | NR2F2 | Human | 7026 |
| cells (venous) | | Mouse | 11819 |
| | NRP2 | Human | 8828 |
| | | Mouse | 18187 |
| Epithelial cells | KRT1 | Human | 3848 |
| (keratinocytes) | | Mouse | 16678 |
| | KRT10 | Human | 3858 |
| | | Mouse | 16661 |
| | KRT14 | Human | 3861 |
| | | Mouse | 16664 |
| Epithelial cells | PMEL (SILV) | Human | 6490 |
| (melanocytes) | | Mouse | 20431 |
| | TYR | Human | 7299 |
| | | Mouse | 22173 |
| | TYRP1 | Human | 7306 |
| | | Mouse | 22178 |
| Bone Cells | BGLAP | Human | 632 |
| (Osteoblasts) | | Mouse | 12096 |
| | COL2A1 | Human | 1280 |
| | | Mouse | 12824 |
| | IBSP | Human | 3381 |
| | | Mouse | 15891 |
| Bone Cells | CALCR | Human | 799 |
| (Osteoclasts) | | Mouse | 12311 |
| | CTSK | Human | 1513 |
| | | Mouse | 13038 |
| Liver Cells | ITGB4 | Human | 3691 |
| (Cholangiocytes) | | Mouse | 192897 |
| | KRT19 | Human | 3880 |
| | | Mouse | 16669 |
| Liver Cells | ALB | Human | 213 |
| (Hepatocytes) | | Mouse | 11657 |
| | G6PC | Human | 2538 |
| | | Mouse | 14377 |
| | TAT | Human | 6898 |
| | | Mouse | 234724 |
| Muscle Cells | MYH6 | Human | 4624 |
| (cardiomyocytes) | | Mouse | 17888 |
| | MYH7 | Human | 4625 |
| | | Mouse | 140781 |
| | NPPA | Human | 4878 |
| | | Mouse | 230899 |
| Muscle Cells (skeletal | CAV3 | Human | 859 |
| muscle cells) | | Mouse | 12391 |
| | MYH1 | Human | 4619 |
| | | Mouse | 17879 |
| | MYOD1 | Human | 4654 |
| | | Mouse | 17927 |
| Muscle Cells | MYH11 | Human | 4629 |
| (smooth muscle cells) | | Mouse | 17880 |
| | SMTN | Human | 6525 |
| | | Mouse | 29856 |
| | TAGLN | Human | 6876 |
| | | Mouse | 21345 |
| Retinal Cells | POU4F2 | Human | 5458 |
| (ganglion cells) | | Mouse | 18997 |
| Retinal Cells | RLBP1 | Human | 6017 |
| (muller cells) | | Mouse | 19771 |
| Retinal Cells | PDE6B | Human | 5158 |
| (photoreceptor cells) | | Mouse | 18587 |
| | RCVRN | Human | 5957 |
| | | Mouse | 19674 |
| Retinal Pigment | PMEL17 | Human | 6490 |
| Epithelial Cells | | Mouse | 20431 |
| | TYRP1 | Human | 7306 |
| | | Mouse | 22178 |
| | BEST1 | Human | 7439 |
| | | Mouse | 24115 |
| | CRALBP | Human | 6017 |
| | | Mouse | 19771 |
| | RPE65 | Human | 6121 |
| | | Mouse | 19892 |
| Renal Cells | NPHS2 | Human | 7827 |
| (podocytes) | | Mouse | 170484 |
| Renal Cells | AQP1 | Human | 358 |
| (proximal tubule | | Mouse | 11826 |
| cells) | CYP27B1 | Human | 1594 |
| | | Mouse | 13115 |
| | MIOX | Human | 55586 |
| | | Mouse | 56727 |
| Renal Cells (collecting | AQP2 | Human | 359 |
| duct cells) | | Mouse | 11827 |
| Renal Cells (distal | UMOD | Human | 7369 |
| tubule cells) | | Mouse | 22242 |
| Adrenal Cells | CYP11A1 | Human | 1583 |
| (cortical cells) | | Mouse | 13070 |
| | HSD3B2 | Human | 3284 |
| | | Mouse | 15493 |
| | FDX1 | Human | 2230 |
| | | Mouse | 14148 |
| Adrenal Cells | PNMT | Human | 5409 |
| (medullary cells) | | Mouse | 18948 |
| | DBH | Human | 1621 |
| | | Mouse | 13166 |
| Pancreatic Cells | GCG | Human | 2641 |
| (alpha cells) | | Mouse | 14526 |
| | MAFB | Human | 9935 |
| | | Mouse | 16658 |
| | POU3F4 | Human | 5456 |
| | | Mouse | 18994 |
| Pancreatic Cells | INS | Human | 3630 |
| (beta cells) | | Mouse | 16334 |
| | MAFA | Human | 389692 |
| | | Mouse | 378435 |
| | SLC2A2 | Human | 6514 |
| | | Mouse | 20526 |
| Pancreatic Cells | SST | Human | 6750 |
| (delta cells) | | Mouse | 20604 |
| Pancreatic Cells | GHRL (Ghrelin, | Human | 51738 |
| (epsilon cells) | Obestatin) | Mouse | 58991 |
| Pancreatic Cells | PPY | Human | 5539 |
| (pancreatic polypeptide | | Mouse | 19064 |
| producing cells) | | | |
| Pancreatic Cells | CPA1 | Human | 1357 |
| (exocrine cells) | | Mouse | 109697 |
| Lung Cells | SFTPB | Human | 6439 |
| | | Mouse | 20388 |
| | SFTPC | Human | 6440 |
| | | Mouse | 20389 |
| | SFTPD | Human | 6441 |
| | | Mouse | 20390 |
| Bone Marrow Cells | CD79A | Human | 973 |
| (early B- | | Mouse | 12518 |
| cell development) | | | |
| Bone Marrow Cells | CD3E | Human | 916 |
| (early T- | | Mouse | 12501 |
| cell development) | PTCRA | Human | 171558 |
| | | Mouse | 19208 |
| Bone Marrow Cells | CCR5 | Human | 1234 |
| (macrophages) | | Mouse | 12774 |
| | CXCR4 | Human | 7852 |
| | | Mouse | 12767 |
| | EMR1 | Human | 2015 |
| | | Mouse | 13733 |
| Bone Marrow Cells | ITGAM | Human | 3684 |
| (monocytes) | | Mouse | 16409 |
| Urothelial Cells | UPK2 | Human | 7379 |
| | | Mouse | 22269 |
| Fibroblasts | COL1A2 | Human | 1278 |
| | | Mouse | 12843 |
| | COL3A1 | Human | 1281 |
| | | Mouse | 12825 |
| Parathyroid Cells | PTH | Human | 5741 |
| | | Mouse | 19226 |
| | CASR | Human | 846 |
| | | Mouse | 12374 |
| Thyroid Cells | NIS | Human | 6585 |
| | | Mouse | 114479 |
| | TSHR | Human | 7253 |
| | | Mouse | 22095 |
| | TPO | Human | 7173 |
| | | Mouse | 22018 |
| | TG | Human | 7038 |
| | | Mouse | 21819 |
| Hypothalamic cells | POMC | Human | 5443 |
| | | Mouse | 18976 |
| | MC4R | Human | 4160 |
| | | Mouse | 17202 |
| Pituitary cells | GH1 | Human | 2688 |
| | | Mouse | 14599 |
| | PRL | Human | 5617 |
| | | Mouse | 19109 |
| | TSHB | Human | 7252 |
| | | Mouse | 22094 |
| | FSHB | Human | 2488 |
| | | Mouse | 14308 |
| | LHB | Human | 3972 |
| | | Mouse | 16866 |
| | PRL | Human | 5617 |
| | | Mouse | 19109 |
| Salivary Gland Cells | PRB1 | Human | 5542 |
| | | Mouse | 381833 |
| | PRH1 | Human | 5554 |
| | | Mouse | 19131 |
| | AMY1A | Human | 276 |
| | | Mouse | 11722 |
| | MUC7 | Human | 4589 |
| | | Mouse | 17830 |
| Ovarian Cells | AMHR2 | Human | 269 |
| | | Mouse | 110542 |
| | FSHR | Human | 2492 |
| | | Mouse | 14309 |
| | CYP19A1 | Human | 1588 |
| | | Mouse | 13075 |
| Testicular Cells | PTGDS | Human | 5730 |
| | | Mouse | 19215 |
| | DLK1 | Human | 8788 |
| | | Mouse | 13386 |

In embodiments where it is desirable to couple the expression of the cell surface binding molecule to the expression of a target cell-specific gene of interest, the recombinant genetic construct is inserted at or around the 3′ untranslated region of any one of the target cell-specific genes described herein. This insertion is achieved using homologous recombination where the recombinant genetic construct is designed to contain 5′ and 3′ “homology alms” referred to herein as first and second nucleotide sequences of a gene expressed in a target cell-specific manner. Thus, the recombinant genetic construct comprises a first nucleotide sequence of a gene expressed in a target cell-specific manner located 5′ to the nucleotide sequence encoding a cell surface molecule, and a second nucleotide sequence of the same gene as the first nucleotide sequence expressed in a target cell-specific manner, where the second nucleotide sequence is located 3′ to the nucleotide sequence encoding the cell surface molecule. These first and second nucleotide sequences of the target cell-specific gene guide the introduction of the recombinant genetic construct into the gene of interest within the cell preparation to be transplanted.

The first and second nucleotide sequences of the target cell-specific gene of the recombinant genetic construct described herein are nucleotide sequences that are the same as or closely homologous (i.e., sharing significant sequence identity) to the nucleotide sequence of particular regions of the target cell-specific gene of interest, i.e., the gene in which the recombinant genetic construct will be inserted into or downstream of Preferably, the first and second nucleotide sequences of the recombinant construct are the same as or similar to the nucleotide sequence of the target cell-specific gene (e.g., the same as the sense strand of the target cell-specific gene) immediately upstream and downstream of an insertion cleavage site.

In some embodiments, the percent identity between the first nucleotide sequence located at the 5′ end of the recombinant construct (i.e., a 5′ homology arm) and the corresponding sequence of target gene (e.g., sense strand) is at least about 70%, 75%, 80%, 85%, 90%, 95%, 98%, 99%, or 100%. In some embodiments, the percent identity between the second nucleotide sequence located at the 3′ end of the recombinant construct (i.e., a 3′ homology arm) and the corresponding sequence of the target gene (e.g., sense strand) is at least about 70%, 75%, 80%, 85%, 90%, 95%, 98%, 99%, or 100%.

In some embodiments, the first and second nucleotide sequences of the target cell-specific gene (e.g., the 5′ and 3′ homology arms) are more than about 30 nucleotide residues in length, for example more than about 50 nucleotide residues, 100 nucleotide residues, 200 nucleotide residues, 300 nucleotide residues, 500 nucleotide residues, 800 nucleotide residues, 1,000 nucleotide residues, 1,500 nucleotide residues, 2,000 nucleotide residues, and 5,000 nucleotide residues in length.

The recombinant genetic construct as disclosed herein may be circular or linear. When the recombinant genetic construct is linear, the first and second nucleotide sequences of the target cell-specific gene (i.e., the 5′ and 3′ homology arms) are proximal to the 5′ and 3′ ends of the linear nucleic acid, respectively, i.e., about 200 bp away from the 5′ and 3′ ends of the linear nucleic acid. In some embodiments, the first nucleotide sequence of the target cell-specific gene (i.e., the 5′ homology arm) is about any of 1, 2, 5, 10, 15, 20, 25, 30, 35, 40, 45, 50, 55, 60, 70, 80, 90, 100, 120, 140, 160, 180, or 200 nucleotide residues away from the 5′ end of the linear DNA. In some embodiments, the second nucleotide sequence of the target cell-specific gene (i.e., the 3′ homology arm) is about any of 1, 2, 5, 10, 15, 20, 25, 30, 35, 40, 45, 50, 55, 60, 70, 80, 90, 100, 120, 140, 160, 180, or 200 nucleotide residues away from the 3′ end of the linear DNA.

The first and second nucleotide sequences of the target cell-specific gene of the recombinant genetic construct are designed to mimic sequences of a target gene expressed in a cell-specific manner to facilitate insertion of the construct into or downstream of the target gene to effectuate tandem expression of the encoded signal incompetent cell surface molecule and the cell-specific gene. The specific location of the insertion site of the genetic construct will vary depending on the target cell-specific gene and, thus, the first and second nucleotide sequences of the target cell-specific gene of the recombinant construct will also vary. However, the selection of these sequences is well within the level of one of skill in the art using the known sequence and structure of the cell-specific gene which is readily available in the art.

The recombinant genetic construct described herein can further comprise one or more nucleotide sequences encoding a reporter molecule that is used for, among other things, in vitro cell identification and/or selection. Suitable markers include, without limitation, enhanced green fluorescent protein (EGFP) or CD4 without cytoplasmic fragment. These markers can be used to determine cell transduction efficiency and/or to select for cells within a population of cells that are expressing the recombinant genetic construct prior to transplantation. Other reporter molecules that can be included in the genetic construct include, without limitation, thymidine kinase, dihydrofolate reductase (together with methotrexate as a DHFR amplifier), aminoglycoside phosphotransferase, hygromycin B phosphotransferase, asparagine synthetase, adenosine deaminase, metallothionein, and antibiotic resistant genes, e.g., the puromycin resistance gene or the neomycin resistance gene. Nucleotide sequences of exemplary antibiotic resistance selection markers are provided in Table 5 below.

TABLE 5
Suitable Selection Marker Gene Sequences

| | | SEQ |
|---|---|---|
| Promoter | | ID |
| Name | Nucleotide Sequence* | NO. |
| Puromycin | ATGACCGAGTACAAGCCCACGGTGCGCCTCGCCACCCGCGAC | 10 |
| Resistance | GACGTCCCCAGGGCCGTACGCACCCTCGCCGCCGCGTTCGCC | |
| | GACTACCCCGCCACGCGCCACACCGTCGATCCGGACCGCCAC | |
| | ATCGAGCGGGTCACCGAGCTGCAAGAACTCTTCCTCACGCGC | |
| | GTCGGGCTCGACATCGGCAAGGTGTGGGTCGCGGACGACGG | |
| | CGCCGCGGTGGCGGTCTGGACCACGCCGGAGGGCGTCGAAG | |
| | CGGGGGCGGTGTTCGCCGAGATCGGCCCGCGCATGGCCGAGT | |
| | TGAGCGGTTCCCGGCTGGCCGCGCAGCAACAGATGGAAGGC | |
| | CTCCTGGCGCCGCACCGGCCCAAGGAGCCCGCGTGGTTCCTG | |
| | GCCACCGTCGGCGTCTCGCCCGACCACCAGGGCAAGGGTCTG | |
| | GGCAGCGCCGTCGTGCTCCCCGGAGTGGAGGCGGCCGAGCG | |
| | CGCCGGGGTGCCCGCCTTCCTGGAGACCTCCGCGCCCCGCAA | |
| | CCTCCCCTTCTACGAGCGGCTCGGCTTCACCGTCACCGCCGAC | |
| | GTCGAGGTGCCCGAAGGACCGCGCACCTGGTGCATGACCCGC | |
| | AAGCCCGGTGCCTGA | |
| Neomycin | ATGAGCCATATTCAACGGGAAACGTCTTGCTCTAGGCCGCGA | 11 |
| Resistance | TTAAATTCCAACATGGATGCTGATTTATATGGGTATAAATGG | |
| | GCTCGCGATAATGTCGGGCAATCAGGTGCGACAATCTATCGA | |
| | TTGTATGGGAAGCCCGATGCGCCAGAGTTGTTTCTGAAACAT | |
| | GGCAAAGGTAGCGTTGCCAATGATGTTACAGATGAGATGGTC | |
| | AGACTAAACTGGCTGACGGAATTTATGCCTCTTCCGACCATC | |
| | AAGCATTTTATCCGTACTCCTGATGATGCATGGTTACTCACCA | |
| | CTGCGATCCCCGGGAAAACAGCATTCCAGGTATTAGAAGAAT | |
| | ATCCTGATTCAGGTGAAAATATTGTTGATGCGCTGGCAGTGTT | |
| | CCTGCGCCGGTTGCATTCGATTCCTGTTTGTAATTGTCCTTTTA | |
| | ACAGCGATCGCGTATTTCGTCTCGCTCAGGCGCAATCACGAA | |
| | TGAATAACGGTTTGGTTGATGCGAGTGATTTTGATGACGAGC | |
| | GTAATGGCTGGCCTGTTGAACAAGTCTGGAAAGAAATGCATA | |
| | AACTTTTGCCATTCTCACCGGATTCAGTCGTCACTCATGGTGA | |
| | TTTCTCACTTGATAACCTTATTTTTGACGAGGGGAAATTAATA | |
| | GGTTGTATTGATGTTGGACGAGTCGGAATCGCAGACCGATAC | |
| | CAGGATCTTGCCATCCTATGGAACTGCCTCGGTGAGTTTTCTC | |
| | CTTCATTACAGAAACGGCTTTTTCAAAAATATGGTATTGATAA | |
| | TCCTGATATGAATAAATTGCAGTTTCATTTGATGCTCGATGAG | |
| | TTTTTCTAA | |
| Hygromycin | ATGAAAAAGCCTGAACTCACCGCGACGTCTGTCGAGAAGTTT | 12 |
| B | CTGATCGAAAAGTTCGACAGCGTCTCCGACCTGATGCAGCTC | |
| | TCGGAGGGCGAAGAATCTCGTGCTTTCAGCTTCGATGTAGGA | |
| | GGGCGTGGATATGTCCTGCGGGTAAATAGCTGCGCCGATGGT | |
| | TTCTACAAAGATCGTTATGTTTATCGGCACTTTGCATCGGCCG | |
| | CGCTCCCGATTCCGGAAGTGCTTGACATTGGGGAGTTCAGCG | |
| | AGAGCCTGACCTATTGCATCTCCCGCCGTGCACAGGGTGTCA | |
| | CGTTGCAAGACCTGCCTGAAACCGAACTGCCCGCTGTTCTCG | |
| | AGCCGGTCGCGGAGGCGATGGATGCGATCGCTGCGGCCGATC | |
| | TTAGCCAGACGAGCGGGTTCGGCCCATTCGGACCGCAAGGAA | |
| | TCGGTCAATACACTACATGGCGTGATTTCATATGCGCGATTG | |
| | CTGATCCCCATGTGTATCACTGGCAAACTGTGATGGACGACA | |
| | CCGTCAGTGCGTCCGTCGCGCAGGCTCTCGATGAGCTGATGC | |
| | TTTGGGCCGAGGACTGCCCCGAAGTCCGGCACCTCGTGCATG | |
| | CGGATTTCGGCTCCAACAATGTCCTGACGGACAATGGCCGCA | |
| | TAACAGCGGTCATTGACTGGAGCGAGGCGATGTTCGGGGATT | |
| | CCCAATACGAGGTCGCCAACATCCTCTTCTGGAGGCCGTGGT | |
| | TGGCTTGTATGGAGCAGCAGACGCGCTACTTCGAGCGGAGGC | |
| | ATCCGGAGCTTGCAGGATCGCCGCGCCTCCGGGCGTATATGC | |
| | TCCGCATTGGTCTTGACCAACTCTATCAGAGCTTGGTTGACGG | |
| | CAATTTCGATGATGCAGCTTGGGCGCAGGGTCGATGCGACGC | |
| | AATCGTCCGATCCGGAGCCGGGACTGTCGGGCGTACACAAAT | |
| | CGCCCGCAGAAGCGCGGCCGTCTGGACCGATGGCTGTGTAGA | |
| | AGTACTCGCCGATAGTGGAAACCGACGCCCCAGCACTCGTCC | |
| | GAGGGCAAAGGAATAG | |

In some embodiments, the recombinant genetic construct further comprises one or more self-cleaving peptide encoding nucleotide sequences, where the self-cleaving peptide encoding nucleotide sequences are positioned within the construct in a manner effective to mediate the translation of the cell surface binding molecule and any other reporter molecules present in the construct. A “self-cleaving peptide” is an 18-22 amino-acid long viral oligopeptide sequence that mediates ribosome skipping during translation in eukaryotic cells (Liu et al., “Systemic Comparison of 2A peptides for Cloning Multi-Genes in a Polycistronic Vector,” Scientific Reports 7: Article Number 2193 (2017), which is hereby incorporated by reference in its entirety). A non-limiting example of such a self-cleaving peptide is Peptide 2A, which is a short protein sequences first discovered in picornaviruses. Peptide 2A functions by making ribosomes skip the synthesis of a peptide bond at the C-terminus of a 2A element, resulting in a separation between the end of the 2A sequence and the peptide downstream thereof. This “cleavage” occurs between the glycine and proline residues at the C-terminus. Thus, successful ribosome skipping and recommencement of translation results in individual “cleaved” proteins where the protein upstream of the 2A element is attached to the complete 2A peptide except for the C-terminal proline and the protein downstream of the 2A element is attached to one proline at the N-terminus (Liu et al., “Systemic Comparison of 2A peptides for Cloning Multi-Genes in a Polycistronic Vector,” Scientific Reports 7: Article Number 2193 (2017), which is hereby incorporated by reference in its entirety).

Exemplary self-cleaving peptides that can be incorporated in the recombinant genetic construct include, without limitation, porcine teschovirus-1 2A (P2A), Foot and mouth disease virus 2A (F2A), those assign virus 2A (T2A), equine rhinitis A virus 2A (E2A), cytoplasmic polyhedrosis virus (BmCPV 2A), and flacherie virus (BmIFV 2A). The nucleotide sequences encoding these self-cleaving peptides that are suitable for inclusion in the recombinant genetic construct described herein are provided in Table 6 below.

TABLE 6
Suitable Self-Cleaving Peptide Coding Nucleotide Sequences

| | | SEQ ID |
|---|---|---|
| Self-Cleaving Peptide | Nucleotide Sequence* | NO. |
| Porcine teschovirus-1 2A (P2A) | GGAAGCGGAG CTACTAACTT | 13 |
| | CAGCCTGCTG AAGCAGGCTG | |
| | GAGACGTGGA GGAGAACCCT GGACCT | |
| Porcine teschovirus-1 2A (P2A), | GGTTCCGGAG CCACGAACTT | 14 |
| codon optimized | CTCTCTGTTA AAGCAAGCAG | |
| | GAGACGTGGA AGAAAACCCC | |
| | GGTCCC | |
| Foot and mouth disease virus 2A | GGAAGCGGAG TGAAACAGAC | 15 |
| (F2A) | TTTGAATTTT GACCTTCTCA | |
| | AGTTGGCGGG AGACGTGGAG | |
| | TCCAACCCTG GACCT | |
| Thosea asigna virus 2A (T2A) | GAGGGCAGAG GAAGTCTTCT | 16 |
| | AACATGCGGT GACGTGGAGG | |
| | AGAATCCCGG CCCT | |
| Equine rhinitis A virus 2A | GGAAGCGGAC AGTGTACTAA | 17 |
| (E2A) | TTATGCTCTC TTGAAATTGG | |
| | CTGGAGATGT TGAGAGCAAC | |
| | CCTGGACCT | |
| Cytoplasmic polyhedrosis virus | GACGTTTTTC GCTCTAATTA | 18 |
| (BmCPV 2A) | TGACCTACTA AAGTTGTGCG | |
| | GTGATATCGA GTCTAATCCT GGACCT | |
| Flacherie virus (BmIFV 2A) | ACTCTGACGA GGGCGAAGAT | 19 |
| | TGAGGATGAA TTGATTCGTG | |
| | CAGGAATTGA ATCAAATCCT GGACCT | |

*See Wang et al., “2A Self-Cleaving Peptide-Based Multi-Gene Expression System in the Silkworm Bombyx mori,” Sci. Rep. 5:16273 (2015) and U.S. Patent Application Publication No. 2018/0369280 to Schmitt et al., which are hereby incorporated by reference in their entirety.

In some embodiments, the recombinant genetic construct further comprises an inducible cell death gene positioned within the construct in a manner effective to achieve inducible cell suicide. An inducible cell death gene refers to a genetically encoded element that allows selective destruction of expressing cells in the face of unacceptable toxicity by administration of an activating pharmaceutical agent.

Several inducible cell death genes are well known in the art and suitable for inclusion in the recombinant genetic construct described herein (see Stavrou et al., “A Rapamycin-Activated Caspase 9-Based Suicide Gene,” Mol. Ther. 26(5):1266-1276 (2018), which is hereby incorporated by reference in its entirety). Exemplary suicide genes include, without limitation, RQR8 and huEGFRt, which are surface proteins recognized by therapeutic monoclonal antibodies (mAbs); herpes simplex virus thymidine kinase (HSV-TK), an inducible cell death gene activated by the small molecule ganciclovir; inducible caspase 9 (iCasp9), a fusion of mutated FKBP12 with the catalytic domain of caspase 9 which allows docking of a small molecular chemical inducer of dimerization (CID, AP1903/AP20187), rapamycin-activated caspase 9 (rapaCasp9), an inducible cell death gene activated by rapamycin (Stavrou et al., “A Rapamycin-Activated Caspase 9-Based Suicide Gene,” Mol. Ther. 26(5):1266-1276 (2018), which is hereby incorporated by reference in its entirety); and inducible caspase-3 (iCasp3), a fusion of mutated FK506 binding domains with caspase-3 which allows docking of a CID (AP20187) (Ono et al., “Exposure to Sequestered Self-Antigens in vivo is not Sufficient for the Induction of Autoimmune Diabetes,” PLos One 12(3):e0173176 (2017) and MacCorkle et al., “Synthetic Activation of Caspases: Artificial Death Switches,” PNAS 95(7): 3655-3660 (1998), which are hereby incorporated by reference in their entirety). In another embodiment, the recombinant genetic construct contains an inducible cell death gene linked to the expression of a cell-division gene, like the cell-division gene (CDK1) (Liang et al., “Linking a Cell-Division Gene and a Suicide Gene to Define and Improve Cell Therapy Safety,” Nature 563:701-704 (2018), which is hereby incorporated by reference in its entirety).

In some embodiments, the recombinant genetic construct of the present disclosure is incorporated into an expression vector. Suitable expression vectors include, without limitation, plasmid vectors, viral vectors, including without limitation, vaccina vectors, lentiviral vector (integration competent or integration-defective lentiviral vectors), adenoviral vectors, adeno-associated viral vectors, vectors for baculovirus expression, transposon based vectors or any other vector suitable for introduction of the recombinant genetic construct described herein into a cell by any means to facilitate the gene/cell selective expression of the recombinant construct.

The systems disclosed herein further include a preparation of one or more cells where cells of the preparation are stably transduced with one or more of the recombinant genetic constructs described herein. In accordance with this aspect, cells of the preparation express at least one of the one or more the recombinant genetic construct as described herein. The cell surface binding molecule encoded by each of the one or more recombinant constructs is not endogenously expressed by the cells of the preparation; however, the surface binding molecule is expressed in a target cell-specific manner either via the activation of the target cell-specific regulatory sequence, e.g., a gene promoter sequence, or expression of the target cell-specific gene.

The preparation of cells may be a preparation of cells from any organism. In some embodiments, the preparation is a preparation of mammalian cells, e.g., a preparation of rodent cells (i.e., mouse or rat cells), rabbit cells, guinea pig cells, feline cells, canine cells, porcine cells, equine cells, bovine cell, ovine cells, monkey cells, or human cells. In one embodiment, the preparation is a preparation of human cells. Suitable cells comprising the recombinant genetic construct as described herein include primary or immortalized embryonic cells, fetal cells, or adult cells, at any stage of their lineage, e.g., totipotent, pluripotent, multipotent, or differentiated cells.

In some embodiments, the preparation is a preparation of pluripotent stem cells. Pluripotent stem cells can give rise to any cell of the three germ layers (i.e., endoderm, mesoderm and ectoderm). In one embodiment, the preparation of cells stably transduced with the recombinant genetic construct is a preparation of induced pluripotent stem cells (iPSCs). In another embodiment, the preparation of cells stably transduced with one or more recombinant genetic constructs is a preparation of pluripotent embryonic stem cells.

In another embodiment, the preparation of one or more cells may be a preparation of multipotent stem cells. Multipotent stem cells can develop into a limited number of cells in a particular lineage. Examples of multipotent stem cells include progenitor cells, e.g., neural progenitor cells which give rise to cells of the central nervous system such as neurons, astrocytes and oligodendrocytes. Progenitor cells are an immature or undifferentiated cell population having the potential to mature and differentiate into a more specialized, differentiated cell type. A progenitor cell can also proliferate to make more progenitor cells that are similarly immature or undifferentiated. Suitable preparations of progenitor cells stably transduced with one or more recombinant genetic constructs include, without limitation, preparations of neural progenitor cells, neuronal progenitor cells, glial progenitor cells, oligodendrocyte-biased progenitor cells, and astrocyte-biased progenitor cells. Other suitable progenitor cell populations include, without limitation, bone marrow progenitor cells, cardiac progenitor cells, endothelial progenitor cells, epithelial progenitor cells, hematopoietic progenitor cells, hepatic progenitor cells, osteoprogenitor cells, muscle progenitor cells, pancreatic progenitor cells, pulmonary progenitor cells, renal progenitor cells, vascular progenitor cells, retinal progenitor cells.

The preparation of cells stably transduced with one or more recombinant genetic constructs as described herein can also be a preparation of differentiated cells. In one embodiment, the preparation of one or more cells may be a preparation of differentiated neurons, oligodendrocytes, or astrocytes. In another embodiment, the preparation of one or more cells expressing one or more recombinant genetic constructs is a preparation of adipocytes, chondrocytes, endothelial cells, epithelial cells (keratinocytes, melanocytes), bone cells (osteoblasts, osteoclasts), liver cells (cholangiocytes, hepatocytes), muscle cells (cardiomyocytes, skeletal muscle cells, smooth muscle cells), retinal cells (ganglion cells, muller cells, photoreceptor cells), retinal pigment epithelial cells, renal cells (podocytes, proximal tubule cells, collecting duct cells, distal tubule cells), adrenal cells (cortical adrenal cells, medullary adrenal cells), pancreatic cells (alpha cells, beta cells, delta cells, epsilon cells, pancreatic polypeptide producing cells, exocrine cells); lung cells, bone marrow cells (early B-cell development, early T-cell development, macrophages, monocytes), urothelial cells, fibroblasts, parathyroid cells, thyroid cells, hypothalamic cells, pituitary cells, salivary gland cells, ovarian cells, and testicular cells.

Additional exemplary cell types that may be stably transduced with one or more recombinant genetic construct described herein include, without limitation, placental cells, keratinocytes, basal epidermal cells, urinary epithelial cells, salivary gland cells, mucous cells, serous cells, von Ebner's gland cells, mammary gland cells, lacrimal gland cells, eccrine sweat gland cells, apocrine sweat gland cells, MpH gland cells, sebaceous gland cells, Bowman's gland cells, Brunner's gland cells, seminal vesicle cells, prostate gland cells, bulbourethral gland cells, Bartholin's gland cells, Littre gland cells, uterine endometrial cells, goblet cells of the respiratory or digestive tracts, mucous cells of the stomach, zymogenic cells of the gastric gland, oxyntic cells of the gastric gland, insulin-producing P cells, glucagon-producing α cells, somatostatin-producing δ cells, pancreatic polypeptide-producing cells, pancreatic ductal cells, Paneth cells of the small intestine, type II pneumocytes of the lung, Clara cells of the lung, anterior pituitary cells, intermediate pituitary cells, posterior pituitary cells, hormone secreting cells of the gut or respiratory tract, gonad cells, juxtaglomerular cells of the kidney, macula densa cells of the kidney, peri polar cells of the kidney, mesangial cells of the kidney, brush border cells of the intestine, striated ducted cells of exocrine glands, gall bladder epithelial cells, brush border cells of the proximal tubule of the kidney, distal tubule cells of the kidney, conciliated cells of the ductulus efferens, epididymal principal cells, epididymal basal cells, hepatocytes, fat cells, type I pneumocytes, pancreatic duct cells, nonstriated duct cells of the sweat gland, nonstriated duct cells of the salivary gland, nonstriated duct cells of the mammary gland, parietal cells of the kidney glomerulus, podocytes of the kidney glomerulus, cells of the thin segment of the loop of Henle, collecting duct cells, duct cells of the seminal vesicle, duct cells of the prostate gland, vascular endothelial cells, synovial cells, serosal cells, squamous cells lining the perilymphatic space of the ear, cells lining the endolymphatic space of the ear, choroid plexus cells, squamous cells of the pia-arachnoid, ciliary epithelial cells of the eye, corneal endothelial cells, ciliated cells having propulsive function, ameloblasts, planum semilunatum cells of the vestibular apparatus of the ear, interdental cells of the organ of Corti, fibroblasts, pericytes of blood capillaries, nucleus pulposus cells of the intervertebral disc, cementoblasts, cementocytes, odontoblasts, odontocytes, chondrocytes, osteocytes, osteoprogenitor cells, hyalocytes of the vitreous body of the eye, stellate cells of the perilymphatic space of the ear, skeletal muscle cells, heart muscle cells, smooth muscle cells, myoepithelial cells, platelets, megakaryocytes, monocytes, connective tissue macrophages, Langerhan's cells, osteoclasts, dendritic cells, microglial cells, neutrophils, eosinophils, basophils, mast cells, plasma cells, helper T cells, suppressor T cells, killer T cells, killer cells, rod cells, cone cells, inner hair cells of the organ of Corti, outer hair cells of the organ of Corti, type I hair cells, cells of the vestibular apparatus of the ear, type II cells of the vestibular apparatus of the ear, type II taste bud cells, olfactory neurons, basal cells of olfactory epithelium, type I carotid body cells, type II carotid body cells, Merkel cells, primary sensory neurons, cholinergic neurons of the autonomic nervous system, adrenergic neurons of the autonomic nervous system, peptidergic neurons of the autonomic nervous system, inner pillar cells of the organ of Corti, outer pillar cells of the organ of Corti, inner phalangeal cells of the organ of Corti, outer phalangeal cells of the organ of Corti, border cells, Hensen cells, supporting cells of the vestibular apparatus, supporting cells of the taste bud, supporting cells of the olfactory epithelium, Schwann cells, satellite cells, enteric glial cells, neurons of the central nervous system, astrocytes of the central nervous system, oligodendrocytes of the central nervous system, anterior lens epithelial cells, lens fiber cells, melanocytes, retinal pigmented epithelial cells, iris pigment epithelial cells, oogonium, oocytes, spermatocytes, spermatogonium, ovarian cells, Sertoli cells, and thymus epithelial cells.

In accordance with this aspect of the disclosure, the recombinant genetic construct is integrated into the chromosome of the one or more cells in the preparation. The term “integrated,” when used in the context of the recombinant genetic construct of the present disclosure means that the recombinant genetic construct is inserted into the genome or the genomic sequence of the one or more cells in the preparation. When integrated, the integrated recombinant genetic construct is replicated and passed along to daughter cells of a dividing cell in the same manner as the original genome of the cell.

Another aspect of the present disclosure relates to an in vivo method of tracking a preparation of transplanted cells in a subject. This method involves providing a system described herein where the system comprises one or more recombinant genetic constructs as described herein, a preparation of cells, wherein cells of the preparation are stably transduced with the one or more recombinant genetic constructs, and one or more radiolabeled binding molecules that bind specifically to the cell surface binding molecule encoded by the one or more recombinant genetic constructs. This method further involves implanting the preparation of cells into the subject, and administering one or more radiolabeled molecules that binds to a cell surface binding molecule encoded by the genetic construct expressed by said preparation of cells sometime after implanting. The method further involves detecting the radiolabeled molecule bound to its cognate cell surface binding molecule expressed by implanted cells of the preparation, thereby non-invasively tracking cells of the preparation in the alive subject.

The preparation of cells may contain one or more recombinant genetic constructs (i.e., one or more different recombinant genetic constructs), whereby expression of a particular recombinant genetic construct, and thus expression of a cell surface molecule is determined by the status of the cell, e.g., the differentiate status of the cell or the identity of the cell. Cells of a preparation, where individual cells contain more than one recombinant genetic construct and each genetic construct has a different reporter cell surface binding molecule, can be distinguished from each other when the reporter molecule is expressed in a cell-specific gene manner, i.e., under the control of a cell-specific gene promoter or expressed in tandem with a cell-specific gene. In this manner, the current identity of the cell or its progeny (which are stably carrying the introduced genetic constructs) can be determined and detected in vivo in the subject, by systemically administering and detecting the radiolabeled molecule bound to its cognate cell surface receptor molecule expressed by a cell of a given identity.

In accordance with this aspect of the disclosure, detecting the radiolabeled molecule bound to its cognate cell surface receptor molecule in a subject can be carried out using molecular imaging with, e.g., positron emission tomography (PET). In alternative embodiments, detecting the radiolabeled molecule bound to its cognate cell surface receptor molecule in a subject can be carried out using Single Photon Emission Computed Tomography (SPECT). In yet another embodiment, detecting the radiolabeled molecule bound to its cognate cell surface receptor molecule in a subject can be carried out using Single Photon Emission Computed Tomography coupled with Computed Tomography (SPECT-CT). Methods of detecting radiolabeled molecules bound to cell surface receptors are known in the art and suitable for use in the methods described herein. See, e.g., Gopal Saha, Fundamentals of Nuclear Pharmacy (2018); Vaquero et al., “Positron Emission Tomography: Current Challenges and Opportunities for Technological Advances in Clinical and Preclinical Imaging Systems,” Annu. Rev. Biomed. Eng. 17:385-414 (2017); Israel et al., “Two Decades of SPECT/CT—the Corning of Age of a Technology; An Updated Review of Literature Evidence,” Eur. J. Nucl. Med. Mol. Imaging 46(10):1990-2012 (2019), which are hereby incorporated by reference in their entirety

In accordance with this aspect of the disclosure, the binding molecule may be radiolabeled with ¹⁵O, ¹¹C, ¹⁸F, ⁶⁴Cu, ⁶⁸Ga, ⁸²Rb, ¹³N, ¹²³I, ^{99m}Tc, or combinations thereof. In some embodiments, the radiolabeled molecule is a molecule that passes the blood-brain barrier. In some embodiments, the radiolabeled molecule is a molecule that does not pass the blood-brain barrier.

Suitable subjects for tracking implanted cells in accordance with the methods of the disclosure include any domesticated or non-domesticated animal. In some embodiments, the subject is a mammal. In some embodiments, the subject is a human. In some embodiments, the subject has a disease or condition warranting a cell transplant.

In accordance with this aspect of the present disclosure, the preparation of cells may be autologous/autogeneic (“self”) to the recipient subject. In another embodiment, the preparation of cells may be non-autologous (“non-self,” e.g., allogeneic, syngeneic, or xenogeneic) to the recipient subject.

Suitable cell preparations are described supra. In some embodiments, the preparation of cells is a preparation of glial progenitor cells. In another embodiment, the preparation of cells is a preparation of bi-potential glial progenitor cells. In one embodiment, the glial progenitor cells can be biased to producing oligodendrocytes. Alternatively, the glial progenitor cells can be biased to producing astrocytes.

Glial progenitor cells can be obtained from embryonic, fetal, or adult brain tissue, embryonic stem cells, or induced pluripotential cells. Suitable methods for obtaining glial progenitor cells from embryonic stem cells or induced pluripotent stem cells are known in the art, see e.g., U.S. Pat. No. 10,450,546 to Goldman and Wang.

Alternatively, the glial progenitor cells are isolated from ventricular and subventricular zones of the brain or from the subcortical white matter. Glial progenitor cells can be extracted from brain tissue containing a mixed population of cells directly by using the promoter specific separation technique, as described in U.S. Patent Application Publication Nos. 20040029269 and 20030223972 to Goldman, which are hereby incorporated by reference in their entirety. This method involves selecting a promoter which functions specifically in glial progenitor cells, and introducing a nucleic acid encoding a marker protein under the control of said promoter into the mixed population cells. The mixed population of cells is allowed to express the marker protein and the cells expressing the marker protein are separated from the population of cells, with the separated cells being the glial progenitor cells.

In some embodiments, cells of a preparation are transduced with a recombinant genetic construct encoding a cell surface binding molecule selected from the dopamine receptor (DRD2), serotonin receptor 4 (HTR4), serotonin receptor 2 (HTR2A), serotonin receptor 1B (HTR1B), dopamine transporter (SLC6A3), and serotonin transporter (SLC6A4). In some embodiments, the preparation of cells is stably transduced with one or more genetic constructs encoding a cell surface binding molecule where the cell surface binding molecule is selected from the dopamine receptor (DRD2), serotonin receptor 4 (HTR4), serotonin receptor 2 (HTR2A), serotonin receptor 1B (HTR1B), dopamine transporter (SLC6A3), and serotonin transporter (SLC6A4), wherein the signal incompetent binding molecule of the one or more constructs is expressed in a cell-specific manner.

In some embodiments, the cell surface binding molecule of the present disclosure is a neuroreceptor target. In some embodiments, the cell surface binding molecule of the present disclosure is a signal incompetent neuroreceptor target. In an embodiment, the cell surface binding molecule is any one of the neuroreceptor targets set forth in Table 7, or combinations thereof. Exemplary central nervous system (CNS) radiotracers for neuroreceptor targets are set forth in Table 7 below, with the number of radioligands continuously being expanded. In an embodiment, the radiolabeled molecule is any one of the radiolabeled molecules set forth in Table 7, or combinations thereof.

TABLE 7
Exemplary CNS Radiotracers
CNS Radiotracers that have been advanced for use in Human Studies

| | | | |
|---|---|---|---|
| | | | ¹²³I-Labeled |
| Molecular Target | ¹¹C-Labeled ligand | ¹⁸F-Labeled ligand | ligand |

Neuroreceptor target
Metabotropic

| | | | |
|---|---|---|---|
| Adenosine A1 | [¹¹C]MPDX | [¹⁸F]CPFPX | |
| Adenosine A2A | [¹¹C]SCH442416 | [¹⁸F]MNI-444 | [¹²³I]MNI-420 |
| | [¹¹C]Preladenant | | |
| | [¹¹C]TMSX | | |
| CB1 | [¹¹C]MePPEP | [¹⁸F]FEMMEP-d2 | |
| | [¹¹C]OMAR | [¹⁸F]MK-9470 | |
| | [¹¹C]SD5024 | | |
| D1 | [¹¹C]NNC 112 | [¹⁸F]MNI-968 ([¹⁸F]PF-8477) | |
| | [¹¹C]SCH 23390 | | |
| D2/D3 | [¹¹C]Raclopride | [¹⁸F]Fallypride | [¹²³I]IBZM |
| | [¹¹C]FLB 457 | | [¹²³I]Epidepride |
| | [¹¹C]MNPA (agonist) | | [¹²³I]IBF |
| | [¹¹C](+)PHNO | | |
| | (agonist) | | |
| | [¹¹C]NPA (agonist) | | |
| H1 | [¹¹C]Doxepin | | |
| H3 | [¹¹C]GSK189254 | [¹⁸F]FMH3 | |
| | [¹¹C]GR 103545 | | |
| 5-HT_{1A} | [carbonyl-¹¹C]WAY | [¹⁸F]FCWAY | |
| | [carbonyl-¹¹C]DWAY | [¹⁸F]MefWAY | |
| | [¹¹C]CUMI-101 | [¹⁸F]MPPF | |
| 5-HT_{1B} | [¹¹C]AZ10419369 | | |
| | [¹¹C]P943 | | |
| 5-HT_{2A} | [¹¹C]MDL 100 907 | [¹⁸F]Altanserin | |
| | [¹¹C]Cimbi-36 | [¹⁸F] MHMZ | |
| | | [¹⁸F]Altanserin-d2 | |
| | | [¹⁸F]Setoperone | |
| 5-HT₄ | [¹¹C]SB-207145 | | |
| 5-HT₆ | [¹¹C]GSK-215083 | | |
| mGluR1 | [¹¹C]ITMM | [¹⁸F]FIMX | |
| mGluR5 | [¹¹C]ABP688 | [¹⁸F]-FPEB | |
| | | [¹⁸F]-PSS232 | |
| NK₁ | | [¹⁸F]SPA-RQ | |
| | | [¹⁸F]MK-0999 ([¹⁸F]FE-SPA- | |
| | | RQ) | |
| NOP | [¹¹C]NOP-1A | | |
| Opiate (DOR) | [¹¹C]Methylnaltrindole | | |
| Opiate (MOR) | [¹¹C]Diprenorphine | [¹⁸F]Fluoroethyldiprenorphine | |
| | [¹¹C]Carfentanil | | |
| | (agonist) | | |
| Opiate (KOR) | [¹¹C]GR103545 | | |
| | [¹¹C]LY2795050 | | |
| Sigma | [¹¹C]SA4503 | | |
| Imidazoline | [¹¹C]BU99008 | | |
| Receptors (I2 | | | |
| binding site) | | | |

Ionotropic

| | | | |
|---|---|---|---|
| Bz(GABAA) | [¹¹C]Flumazenil | [¹⁸F]Flumazenil | |
| Bz (α5GABAA) | [¹¹C]Ro15 4513 | | |
| Nicotinic (α4β2) | | 2-[¹⁸F]F-A-85380 (2-[¹⁸F]FA) | [¹²³I]51A |
| | | 6-[¹⁸F]FA | |
| | | [¹⁸F]Nifene (agonist) | |
| | | [¹⁸F]XTRA | |
| | | [¹⁸F]GMOM | |
| | | [¹⁸F]Flubatine | |
| | | [¹⁸F]AZAN | |
| Nicotinic (α7) | [¹¹C]CHIBA-1001 | [¹⁸F]ASEM | |
| NMDA | | [¹⁸F]GE-179 | [¹²³I]CNS1261 |
| P2X7 | [¹¹C]JNJ54173717 | [¹⁸F]JNJ-64413739 | |
| | [¹¹C]GSK-1482160 | | |

Transporter target

| | | | |
|---|---|---|---|
| DAT | [¹¹C]PE2I | [¹⁸F]FP-CIT | [¹²³I]FP-CIT |
| | | | (DATSCAN) |
| | [¹¹C]Methylphenidate | [¹⁸F]FE-PE2I | [¹²³I]CIT |
| | | | (Dopascan) |
| | | [¹⁸F]FECNT | [¹²³I]Altropane |
| | | | [¹²³I]PE2I |
| Glycine T1 | [¹¹C]CFpyPB | [¹⁸F]CFPyPB | |
| | [¹¹C]GSK 931145 | | |
| | [¹¹C]RO5013853 | | |
| NET | [¹¹C]MeNER-d2 | [¹⁸F]FMeNER-d2 | [¹²³I]INER |
| SERT | [¹¹C]DASB | [¹⁸F]ADAM | [¹²³I]CIT |
| | [¹¹C]MADAM | | [¹²³I]mZIENT |
| | [¹¹C]AFM | | [¹²³I]norβCIT |
| | [¹¹C]HOHMADAM | | [¹²³I]βCIT |
| | | | [¹²³I]ADAM |
| VMAT2 | [¹¹C]DTBZ | [¹⁸F]florbenazine | |
| | [¹¹C]MTBZ | [¹⁸F]AV-133 | |
| | | [¹⁸F]FP-DTBZ | |
| VAChT | | [¹⁸F]FEOBV | |

Synaptic Proteins

| | | |
|---|---|---|
| SV2A | [¹¹C]UCB-J | [¹⁸F]UCB-H |
| | [¹¹C]UCB-A | |

Channel like target

| | | | |
|---|---|---|---|
| TSPO | [¹¹C](R)-PK 11195 | [¹⁸F]FBR | [¹²³I]CLINDE |
| | [¹¹C]PBR28 | [¹⁸F]FEPPA | |
| | [¹¹C]DAA1106 | [¹⁸F]PBR111 | |
| | [¹¹C]DPA-713 | | |

Enzyme target

| | | | |
|---|---|---|---|
| AChE | [¹¹C]MP4A | | [¹²³I]IBVM |
| Aromatase | [¹¹C]VOR | | |
| Cox-1 | [¹¹C]PS13 | | |
| FAAH | [¹¹C]CURB | | |
| | [¹¹C]MK3168 | | |
| HDAC 1-3 | [¹¹C]Martinostat | | |
| MAO-A | [¹¹C]Harmine | | |
| | [¹¹C]Clorgyline | | |
| | [¹¹C]Befloxatone | | |
| MAO-B | [¹¹C]Deprenyl-d2 | | |
| Mitochondrial | | [¹⁸F]BCPP-EF | |
| Complex 1 | | | |
| PDE2A | | [¹⁸F]PF05270430 | |
| PDE4 | [¹¹C](R)-Rolipram | | |
| PDE10A | [¹¹C]IMA107 | [¹⁸F]MNI659 | |
| | [¹¹C]MP-10 | | |
| | [¹¹C]Lu AE92686 | | |

Aggregated protein target

| | | | |
|---|---|---|---|
| β-Amyloid | [¹¹C]PIB | [¹⁸F]Flutemetamol | [¹²³I]IMPY |
| | | [¹⁸F]Florbetapir([¹⁸F]AV-45) | |
| | | [¹⁸F]AZD 4694 | |
| | | [¹⁸F]FBM | |
| | | [¹⁸F]FDDNP | |
| | | [¹⁸F]W372 | |
| | | [¹⁸F]Florbetaban | |
| | | [¹⁸F]MK3328 | |
| Tau/Synuclein | [¹¹C]PBB3 | [¹⁸F]BF-227 | |
| | [¹¹C]THK5351 | [¹⁸F]Flortaucipir ([¹⁸F]AV- | |
| | | 1451) | |
| | | [¹⁸F]THK5351 | |
| | | [¹⁸F]THK5317 | |
| | | [¹⁸F]THK5105 | |
| | | [¹⁸F]THK523 | |
| | | [¹⁸F]MK6240 | |
| | | [¹⁸F]RO948 | |

Source: The National Institute of Mental Health (2020)

In some embodiments, the cell surface binding molecule is a dopamine receptor, and the radiolabeled molecule is a selective dopamine agonist or antagonist labeled with ¹¹C, ¹²¹I, or ¹⁸F. Exemplary radiolabeled dopamine receptor ligands include, without limitation, those provided in Table 7 above. In some embodiments, the radiolabeled dopamine receptor ligand is selected from [¹¹C]-racloprid, 3-N-(2-[¹⁸F]-fluoroethyl)-spiperone, [¹¹C]-SCH23990, and [¹⁸F]-fallypride

In some embodiments, the cell surface binding molecule is a serotonin receptor, and the radiolabeled molecule is a selective serotonin agonist or antagonist labeled with ³H, ¹¹C, ¹²³I, or ¹⁸F. Exemplary radiolabeled serotonin receptor ligands include, without limitation, those provided in Table 7 above. In some embodiments, the radiolabeled serotonin receptor ligand is selected from [¹¹C]AZ10419369 (serotonin receptor 11B), [¹¹C]P943 (serotonin receptor 11B), [¹¹C]Cimbi-36 (serotonin receptor 2), [¹⁸F]Altanserin (serotonin receptor 2), [¹¹C](R)-M100907 (serotonin receptor 2), [¹¹C]SB207145 (serotonin receptor 4).

Another aspect of the present disclosure relates to a preparation of cells, wherein the cells of the preparation are stably transduced with one or more recombinant genetic construct, each genetic construct comprising a cell-specific gene promoter and a nucleotide sequence encoding a cell surface binding molecule, wherein said nucleotide sequence is positioned 3′ to the cell-specific gene promoter, and wherein the cell surface binding molecule is not endogenously expressed by cells of the preparation.

Another aspect of the present disclosure relates to a preparation of cells, wherein the cells of the preparation are genetically modified with one or more recombinant genetic constructs, each construct comprising a first nucleotide sequence of a gene expressed in a target cell-specific manner, a cell surface binding molecule encoding nucleic acid molecule, wherein the nucleotide sequence is positioned 3′ to the first nucleotide sequence of the recombinant construct and wherein the cell surface binding molecule is not endogenously expressed by cells of said preparation, and a second nucleotide sequence from the same gene as the first nucleotide sequence expressed in the target cell-specific gene, where the second nucleotide sequence is located 3′ to the nucleotide sequence encoding the cell surface binding molecule.

Suitable cells, genetic constructs, and cell surface binding molecules of the preparations of cells are described above.

### EXAMPLES

The examples below are intended to exemplify the practice of embodiments of the disclosure but are by no means intended to limit the scope thereof.

### Example 1— Expression Levels of Selected Receptors in HAD100 Cells

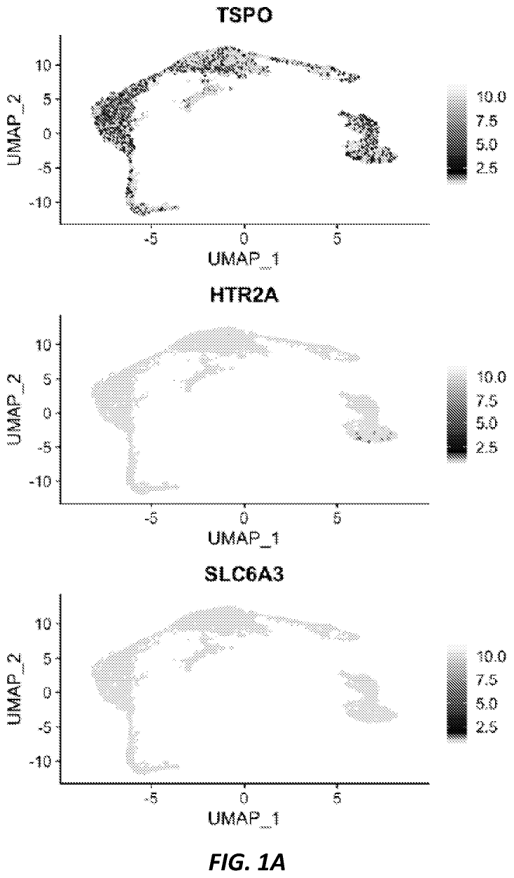

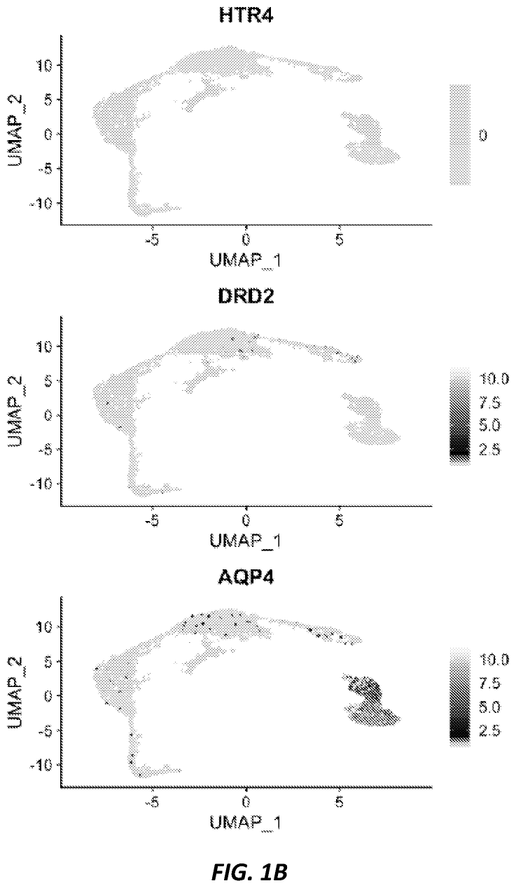

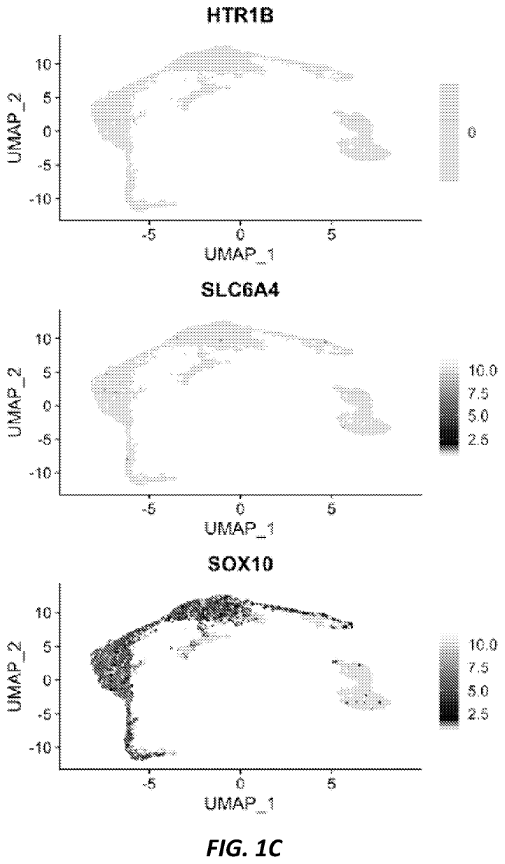

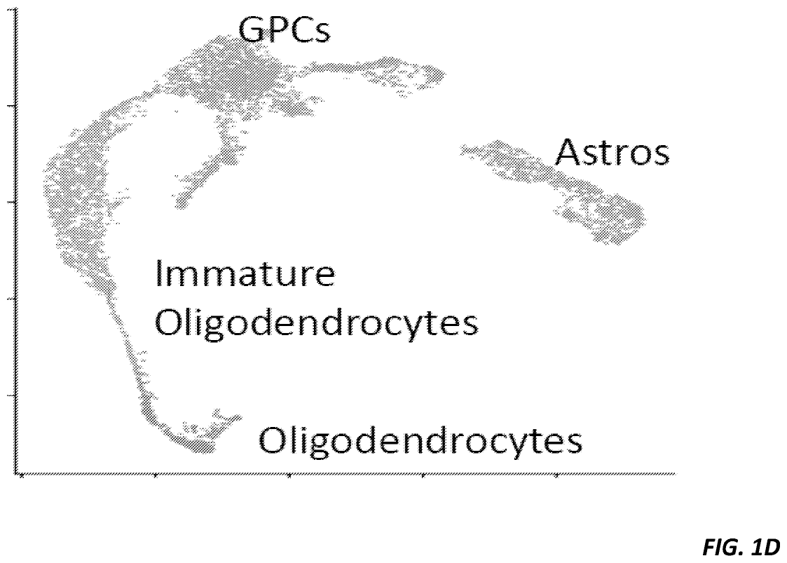

The UMAP method is a manifold learning technique for dimension reduction (see, e.g., in Becht et al., “Dimensionality Reduction for Visualizing Single-Cell Data using UNIAP,” Nature Biotechnology 37:38-44 (2019), which is hereby incorporated by reference in its entirety). A uniform manifold approximation and projection of single-cell RNAseq expression levels of selected receptors in HAD100 cells colored according to cell population (i.e., astrocytes (Astros), glial progenitor cells (GPCs), Immature Oligodendrocytes, and Oligodendrocytes) is shown in FIG. 1D. The expression levels of TSPO, HTR2A, SLC6A3, HTR4, DRD2, HTR1B, SLC6A4, AQP4, and SOX10 for the identified cell populations (i.e., astrocytes (Astros), glial progenitor cells (GPCs), Immature Oligodendrocytes, and Oligodendrocytes) are shown in FIGS. 1A-1C.

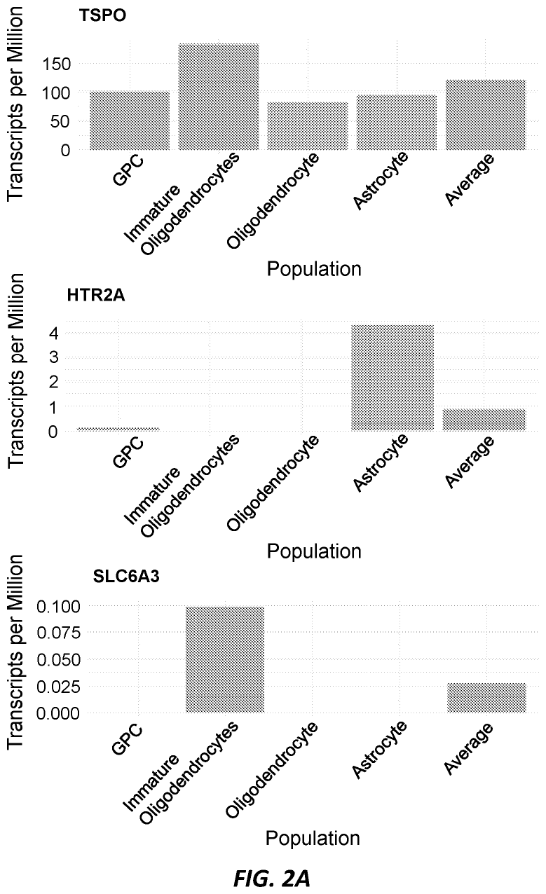

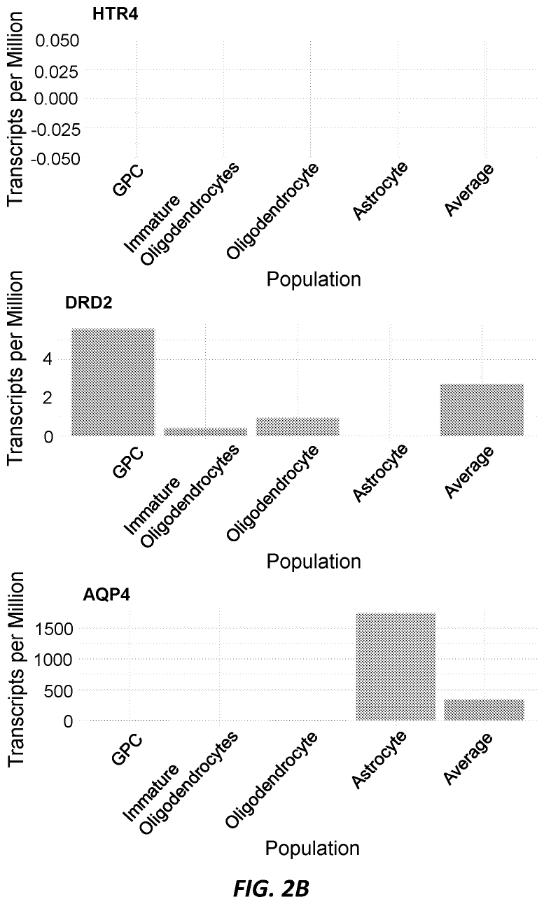

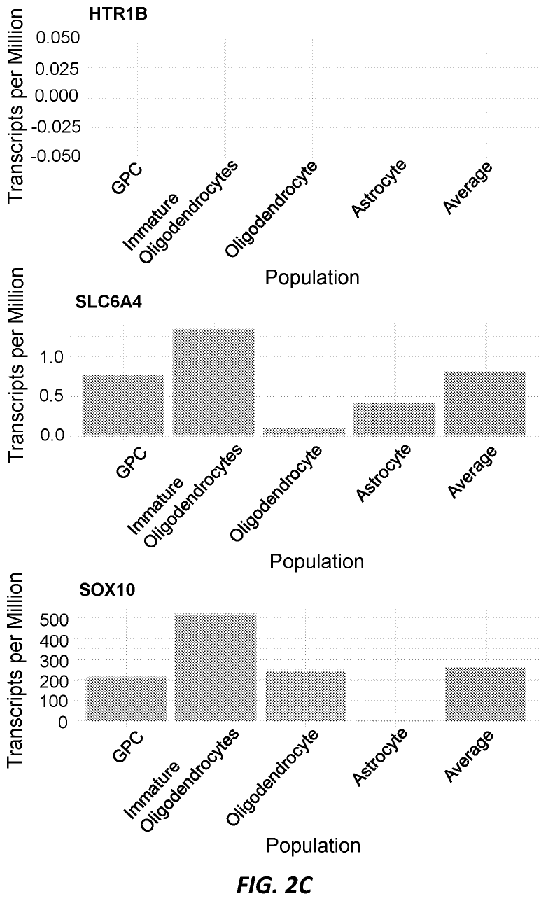

The expression levels (in transcripts per million) of the receptors identified in FIGS. 1A-1C (i.e., TSPO, HTR2A, SLC6A3, HTR4, DRD2, HTR1B, SLC6A4, AQP4, and SOX10) by cell population (i.e., astrocytes (Astros), glial progenitor cells (GPCs), Immature Oligodendrocytes, and Oligodendrocytes) is shown in FIGS. 2A-2C.

The results presented in FIGS. 1A-1C and FIGS. 2A-2C identify various receptors that may be used or excluded from use in the methods disclosed herein. For example, HTR2A is expressed in populations of astrocytes, but has low expression levels in glial progenitor cells, immature oligodendrocytes, and oligodendrocytes (FIG. 1A and FIG. 2A). In contrast, the results in FIG. 1A and FIG. 2A demonstrate that transcripts for the TPSO gene are expressed at greater than 50 transcripts per million in populations of astrocytes, glial progenitor cells, oligodendrocytes, and immature oligodendrocytes. Moreover, the results in FIGS. 1B-1C and FIGS. 2B-2C show that transcripts for HTR4 and HTR1B are not detected in any of the cell populations evaluated (i.e., populations of astrocytes, glial progenitor cells, oligodendrocytes, and immature oligodendrocytes).

### Example 2—Design of Cell Surface Binding Molecules

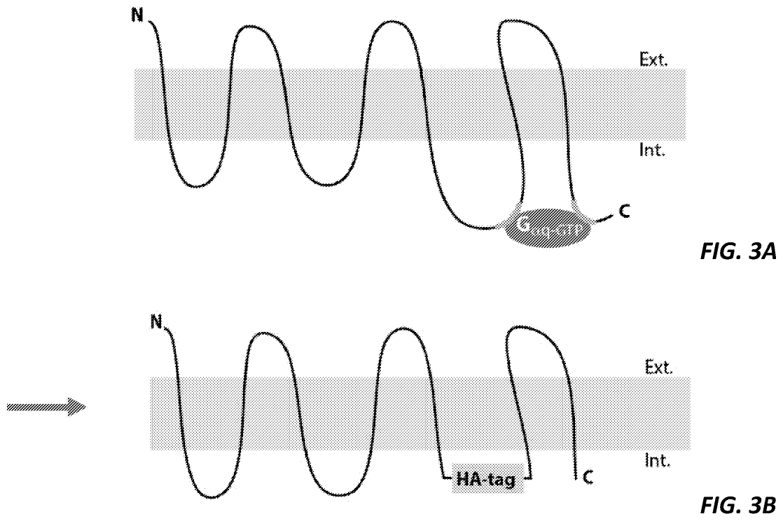

As described herein above, a suitable cell surface binding molecule for use in the methods of the present application may be designed by modifying, removing, or replacing an intercellular fragment necessary to transmit an extracellular ligand binding event to the intercellular space. For example, the G protein binding site may be modified by one or more amino acid substitutions, insertions, or deletions. Suitable cell surface binding molecules for use in the methods of the present application may be identified, e.g., by carrying out UMAP analysis as described in Example 1. As shown in FIGS. 3A-3B, the G protein binding site (G_{oq-GTP}) may be modified by replacement of the binding site (FIG. 3A), or at least a portion of the binding site, with a sequence that is incapable of transmitting a signal, e.g., an HA tag comprising the amino acid sequence YPYDVPDYA (SEQ ID NO:1) (FIG. 3B).

### Example 3—Recombinant Genetic Constructs Comprising Cell-Type Specific Promoters

Recombinant genetic constructs comprising cell-type specific promoters were designed to include: (i) a regulatory sequence driving target cell-type specific gene expression and (ii) a nucleotide sequence encoding a cell surface binding molecule, where the nucleotide sequence is positioned 3′ to the regulatory sequence driving cell-type specific gene expression of the recombinant genetic construct.

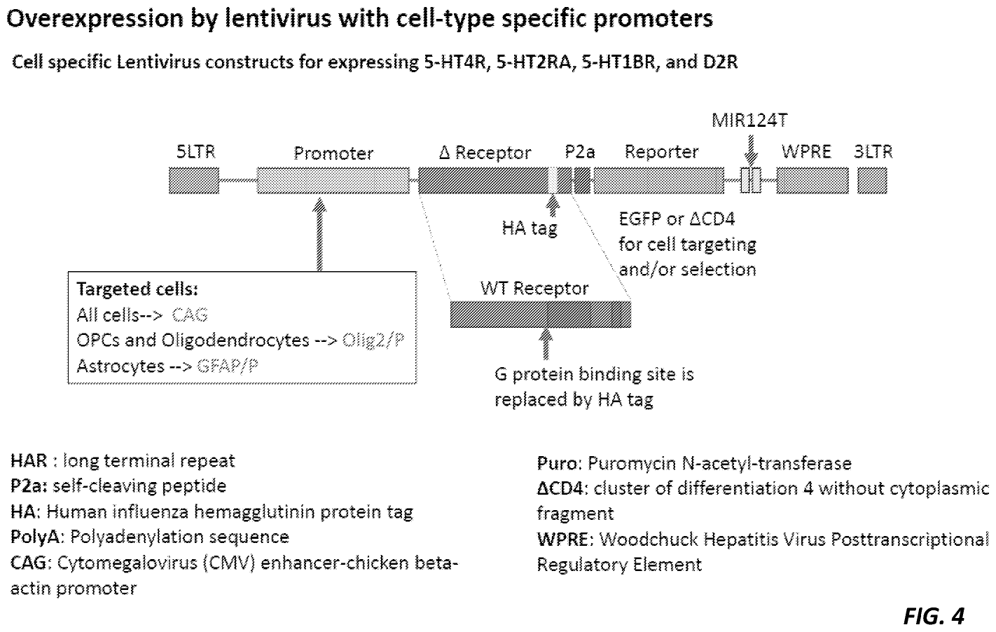

FIG. 4 shows a schematic of a cell-type specific recombinant construct comprising a cell-type specific promoter (e.g., Cytomegalovirus enhancer-chicken beta-actin promoter; Olig2/P, or GFAP/P) for regulating expression of a cell surface binding molecule (A Receptor) in specific cell populations. As shown in FIG. 4, the cytomegalovirus (CVM) enhancer-chicken beta-actin promoter (CAG) can be used to target all cells, the Olig2 promoter can be used to target OPCs and oligodendrocytes, and the GFAP promoter can be used to target astrocytes. The recombinant genetic construct shown in FIG. 4 is a lentivirus construct suitable for expressing a cell surface binding molecule (e.g., a modified 5-HT4R, 5-HT2RA, 5-HT1BR, or D2R as described herein) in a target cell population. Moreover, as shown in FIG. 4, the recombinant genetic construct is designed to comprise, 5′→3′, a homology arm right (HAR) consisting of a 5′ long terminal repeat (LTR) region (5LTR), a cell-type specific promoter (Promoter), a cell surface binding molecule comprising a HA tag (Δ Receptor), a self-cleaving peptide (P2a), an enhanced green fluorescent protein (EGFP) or cluster of differentiation 4 without cytoplasmic fragment (ΔCD4) for cell targeting and/or selection (Reporter), a microRNA124 target sequence (MIR124T), the woodchuck hepatitis virus posttranscriptional regulatory element (WPRE), and a homology arm left consisting of a 3′ LTR region (3LTR).

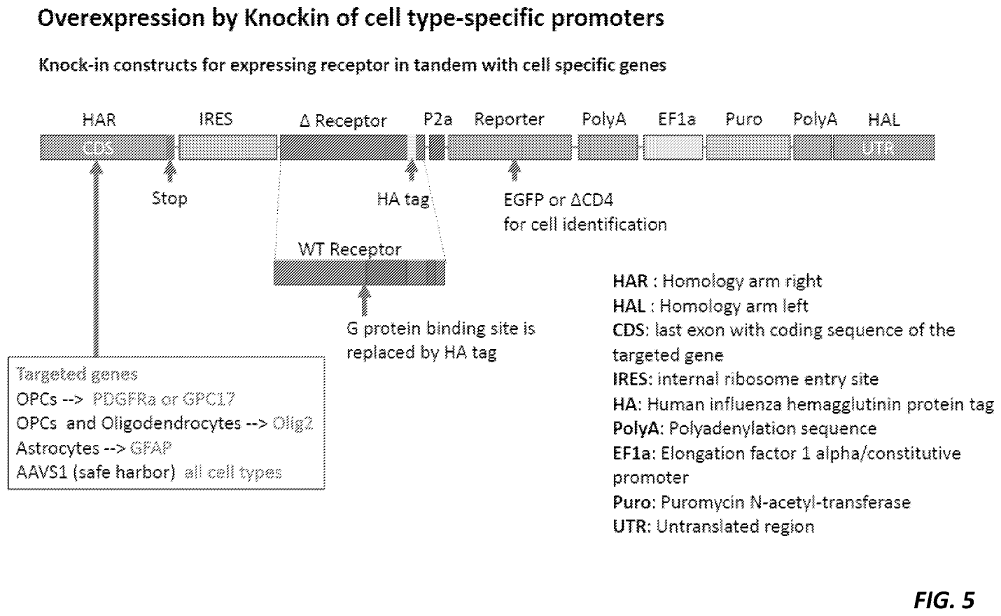

FIG. 5 shows a schematic of a knock-in construct for expressing a cell surface binding molecule in a cell-specific manner, where the cell surface binding molecule (e.g., a modified 5-HT4R, 5-HT2RA, 5-HT1BR, or D2R as described herein) is expressed in tandem with a gene expressed in a target cell-specific manner. As shown in FIG. 5, the recombinant genetic construct is designed to comprise, 5′→3′, a right homology arm (HAR), (i.e., a first nucleotide sequence of a gene expressed in a target cell-specific manner); an internal ribosome entry site (IRES); a cell surface binding molecule comprising a HA tag (Δ Receptor) (i.e., signal incompetent cell surface binding molecule of the present application); a self-cleaving peptide (P2a); an enhanced green fluorescent protein (EGFP) or cluster of differentiation 4 without cytoplasmic fragment (ΔCD4) for cell targeting and/or selection (Reporter); a first polyadenylation sequence (PolyA); an elongation factor 1 alpha/constitutive promoter (EF1a); a puromycin N-acetyl-transferase (Puro); a second polyadenylation sequence (PolyA); and a left homology arm (HAL) (i.e., a second nucleotide sequence from the same gene as the first nucleotide sequence expressed in the target cell-specific manner). The recombinant genetic construct shown in FIG. 5 may be used to target (i) the AAVS1 gene (which is a known safe harbor for hosting DNA transgenes with expected function), which can be used to target all cell types; (ii) the platelet-derived growth factor receptor A (PDGFRa) or GPR17 genes to target OPCs; (iii) the Olig2 gene can be used to target OPCs and oligodendrocytes; and (iv) the GFAP gene to target astrocytes.

### Example 4—Expression of Δ-Drd2 Receptor in Mice Transduced with a Lentivirus Construct Expressing LV-Δ-Drd2

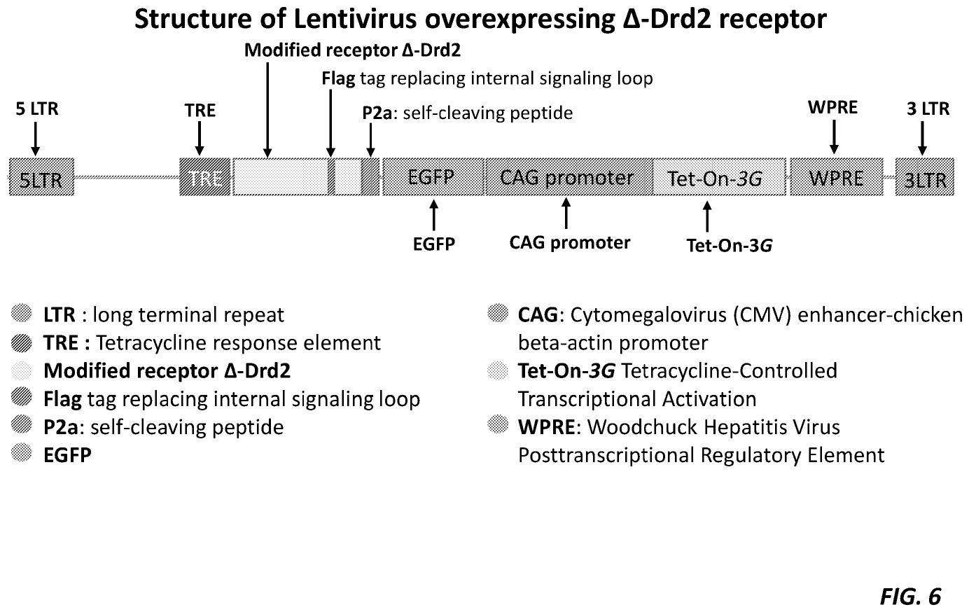

A recombinant lentivirus construct comprising a nucleotide sequence encoding A-dopamine receptor D2 (Drd2) (i.e., a cell surface binding molecule) was designed to include a 5′ long terminal repeal (5LTR), a tetracycline response element (TRE), the nucleotide sequence encoding the modified receptor Δ-Drd2 (i.e., signal incompetent cell surface binding molecule), a nucleotide sequence encoding P2a (i.e., a self-cleaving peptide), a nucleotide sequence encoding enhanced green fluorescent protein (EGFP) (i.e., a reporter), a cytomegalovirus (CMV) enhancer-chicken beta-actin promoter (CAG Promoter), a tetracycline-controlled transcriptional activator (Tet-On-3G), a woodchuck hepatitis virus posttranscriptional regulatory element (WPRE), and a 3′ long terminal repeat (3LTR). A schematic of the Δ-Drd2 portion of the lentivirus construct is provided in FIG. 6.

LV-Δ-Drd2 virus particles were pseudotyped with vesicular stomatitis virus G glycoprotein envelope. High titer was produced by transient transfection of HEK-293FT and concentrated by ultracentrifugation. The virus was titrated by QPCR (1.5×10⁸ IU/ml).

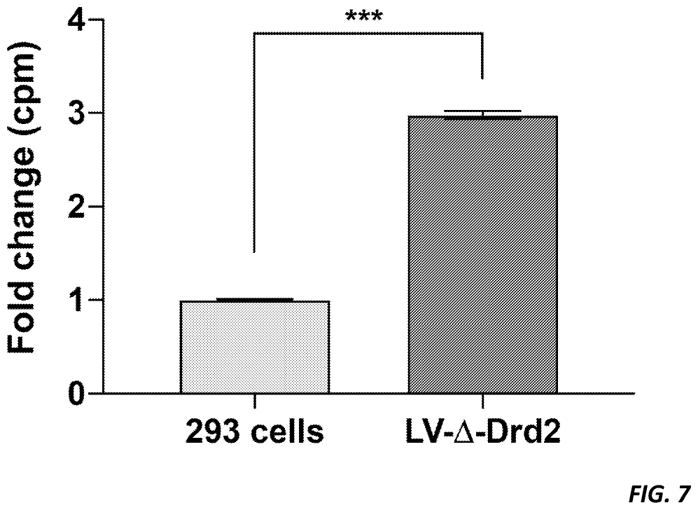

To assess in vitro ligand binding to the signal-inactivated receptor, HEK-293FT cells were infected with lentivirus expressing LV-Δ-Drd2 (MOI=1) or untreated. Equal cell numbers were treated with H₃-Raclopride for 1 hour at 37° C.; (n=3 each). One hour later the cells were collected and resuspended in scintillation counter for detection of the amount of radioligand binding (FIG. 7).

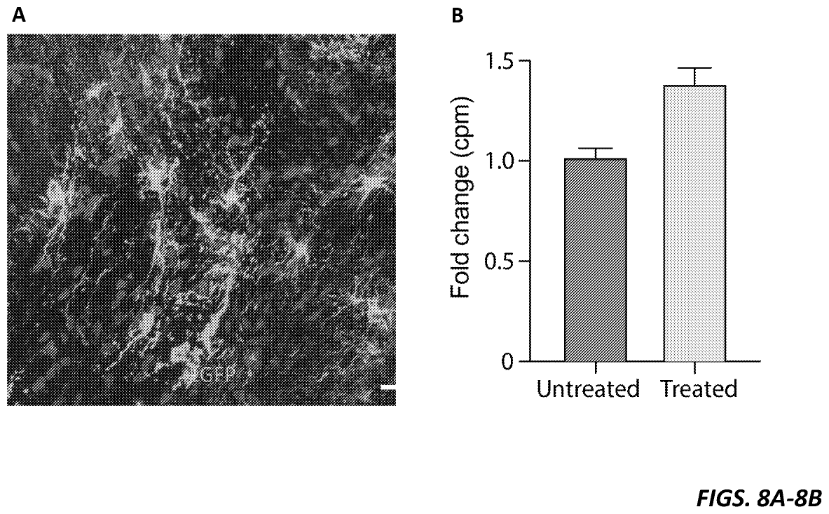

To assess in vivo ligand binding to the signal-inactivated receptor as expressed by lentivirus, eight-week old mice received stereotaxic intra-striatal injection of 1 μl lentivirus expressing LV-Δ-Drd2 (treated hemisphere) or sham (untreated hemisphere). One week later the mice were either sacrificed and their brains processed for histology to confirm transduction with lentivirus (FIG. 8A), or injected with H₃-Raclopride (IV, 1 μCi in 100 μl saline). The latter were sacrificed 10 minutes after radioligand injection, and their striata dissected and assessed by scintillation counter for radioligand binding to the expressed dopamine receptor D2 (FIG. 8B). While mice striatum express wildtype dopamine receptor D2, the increase in radioligand binding above this baseline demonstrated definitive in vivo ligand binding to the signal-inactivated receptor expressed by the lentivirus.

Although preferred embodiments have been depicted and described in detail herein, it will be apparent to those skilled in the relevant art that various modifications, additions, substitutions, and the like can be made without departing from the spirit of the invention and these are considered to be within the scope of the invention as defined in the following claims.

What is claimed:

1. A system for in vivo tracking of target cells resulting from implantation of a preparation of cells, said system comprising:
one or more recombinant genetic constructs, each construct comprising:
a regulatory sequence driving target cell-type specific gene expression; and
a nucleotide sequence encoding a cell surface binding molecule, wherein said nucleotide sequence is positioned 3′ to the regulatory sequence driving target cell-type specific gene expression of the recombinant genetic construct;
a preparation of cells, wherein cells of the preparation are stably transduced with the one or more recombinant genetic constructs, wherein the cell surface binding molecule encoded by each of the one or more recombinant constructs is not endogenously expressed by said cells of the preparation, and whereby the regulatory sequence driving target cell-type specific gene expression is activated when present in the target cell to express the cell surface binding molecule in the target cell; and
one or more radiolabeled binding molecules that bind to the cell surface binding molecule encoded by the one or more recombinant genetic constructs.

2. The system of claim 1, wherein the regulatory sequence driving target cell-type specific gene expression is from a gene that is restrictively expressed in one or more differentiated cell types.

3. The system of claim 2, wherein the differentiated cell is an oligodendrocyte.

4. The system of claim 3, wherein the regulatory sequence driving target cell-type specific gene expression is from a gene selected from the group consisting of SRY-box 10 (SOX10), Myelin Regulatory Factor (MYRF), Myelin-associated Glycoprotein (MAG), and Myelin Basic Protein (MBP).

5. The system of claim 2, wherein the differentiated cell is an astrocyte.

6. The system of claim 5, wherein the regulatory sequence driving target cell-type specific gene expression is from a gene selected from glial fibrillary acidic protein (GFAP) and aquaporin-4 (AQP4).

7. The system of claim 2, wherein the differentiated cell is a neuron.

8. The system of claim 7, wherein the regulatory sequence driving target cell-type specific gene expression is from a gene selected from the group consisting of synapsin 1 (SYN1), microtubule associated protein 2 (MAP2), and ELAV like RNA binding protein 4 (ELAV4).

9. The system of claim 2, wherein the differentiated cell is a dopaminergic neuron and the regulatory sequence driving target cell-type specific gene expression is from the tyrosine hydroxylase (TH) gene or the DOPA decarboxylase (DDC) gene.

10. The system of claim 2, wherein the differentiated cells are medium spiny neurons and cortical interneurons and the regulatory sequence driving target cell-type specific gene expression is from glutamate decarboxylase 2 (GAD2/GAD65) or glutamate decarboxylase 1 (GAD1/GAD67).

11. The system of claim 2, wherein the differentiated cell is a cholinergic neuron and the regulatory sequence driving target cell-type specific gene expression is from choline O-acetyltransferase (CHAT).

12. The system of claim 1, wherein the regulatory sequence driving target cell-type specific gene expression is from a gene that is restrictively expressed in a progenitor cell type.

13. The system of claim 12, wherein the progenitor cell is a glial progenitor cell and the regulatory sequence driving target cell-type specific gene expression is from a gene selected from platelet derived growth factor receptor α (PDGFRα), CD44, or oligodendrocyte transcription factor 2 (OLIG2).

14. A system for in vivo tracking of target cells resulting from implantation of cells comprising:
one or more recombinant genetic constructs, each construct comprising:
a first nucleotide sequence of a gene expressed in a target cell-specific manner;
a cell surface binding molecule encoding nucleotide sequence, wherein said nucleotide sequence is positioned 3′ to the first nucleotide sequence of the recombinant genetic construct; and
a second nucleotide sequence from the same gene as the first nucleotide sequence expressed in the target cell-specific manner, said second nucleotide sequence located 3′ to the cell surface binding molecule encoding nucleotide sequence;
a preparation of cells, wherein cells of the preparation are genetically modified with the one or more recombinant genetic constructs to express the cell surface binding molecule in tandem with the gene expressed in the target cell-specific manner, wherein the cell surface binding molecule is not endogenously expressed by said target cells; and
one or more radiolabeled binding molecules that bind to the cell surface binding molecule encoded by the one or more recombinant genetic constructs.

15. The system of claim 14, wherein the first and second nucleotide sequences of the recombinant genetic construct are from a gene that is restrictively expressed in one or more differentiated cell types.

16. The system of claim 15, wherein the differentiated cell is an oligodendrocyte.

17. The system of claim 16, wherein the first and second nucleotide sequences of the recombinant genetic construct are from a gene selected from the group consisting of SOX10, MYRF, MAG, and MBP.

18. The system of claim 15, wherein the differentiated cell is an astrocyte.

19. The system of claim 18, wherein the first and second nucleotide sequences of the recombinant genetic construct are from a gene selected from GFAP and AQP4.

20. The system of claim 15, wherein the differentiated cell is a neuron.

21. The system of claim 20, wherein the first and second nucleotide sequences of the recombinant genetic construct are from a gene selected from the group consisting of SYN1, MAP2, and ELAV4.

22. The system of claim 15, wherein the terminally differentiated cell is a dopaminergic neuron and the first and second nucleotide sequences of the recombinant genetic construct are from TH or DDC.

23. The system of claim 15, wherein the differentiated cells are medium spiny neurons and cortical interneurons and the first and second nucleotide sequences of the recombinant genetic construct are from GAD65 or GAD67.

24. The system of claim 15, wherein the differentiated cell is a cholinergic neuron and the first and second nucleotide sequences of the recombinant genetic construct are from CHAT.

25. The system of claim 15, wherein the first and second nucleotide sequences of the recombinant genetic construct are from a gene that is restrictively expressed in a progenitor cell.

26. The system of claim 25, wherein the progenitor cell is a glial progenitor cell and the first and second nucleotide sequences of the recombinant genetic construct are from a gene selected from platelet derived growth factor receptor α (PDGFRα), CD44, and oligodendrocyte transcription factor 2 (OLIG2).

27. The system of any one of claims 1-26, wherein cell surface binding molecule is selected from a cell surface receptor, a glycoprotein, a cell adhesion molecule, an antigen, an integrin, or a cluster of differentiation (CD).

28. The system of claim 27, wherein the cell surface binding molecule is a cell surface receptor.

29. The system of claim 28, wherein the cell surface receptor is a signal incompetent form of the cell surface receptor.

30. The system of claim 29, wherein cell surface receptor is selected from the group consisting of a signal incompetent form of a dopamine receptor (DRD2), a signal incompetent form of a serotonin receptor 4 (HTR4), a signal incompetent form of a serotonin receptor 2 (HTR2A), a signal incompetent form of a serotonin receptor 1B (HTR1B), a signal incompetent form of a dopamine transporter (SLC6A3), and a signal incompetent form of a serotonin transporter (SLC6A4).

31. The system of any one of claims 1-30, wherein the radiolabeled binding molecule is labeled with ¹²³I, ^{99m}Tc, ¹¹C, or ¹⁸F.

32. The system of claim 31, wherein the cell surface binding molecule is a cell surface receptor and the radiolabeled binding molecule is the cell surface receptor's ligand.

33. The system of any one of claims 1-32, wherein said recombinant genetic construct of the system further comprises a nucleotide sequence encoding a reporter molecule.

34. The system of claim 33, wherein the reporter molecule is EGFP or a signal incompetent CD4.

35. The system of any one of claims 1-34, wherein the recombinant genetic construct of the system further comprises:
one or more self-cleaving peptide encoding nucleotide sequences, wherein said self-cleaving peptide encoding nucleotide sequence is positioned within the construct in a manner effective to separate translation of the cell surface binding molecule and the reporter molecule.

36. The system of claim 35, wherein the self-cleaving peptide is selected from the group consisting of porcine teschovirus-1 2A (P2A), those assign virus 2A (T2A), equine rhinitis A virus 2A (E2A), cytoplasmic polyhedrosis virus (BmCPV 2A), and flacherie virus (BmIFV 2A).

37. The system of any one of claims 1-36, wherein the recombinant genetic construct of the system further comprises:
an inducible cell death gene positioned within the construct in a manner effective to achieve inducible cell suicide.

38. The system of claim 37, wherein the inducible cell death gene is selected from caspase-3, caspase-9, and thymidine kinase.

39. The system of any one of claims 1-38, wherein said recombinant genetic construct is in an expression vector.

40. The system of claim 39, wherein the expression vector is a viral vector, plasmid vector, or bacterial vector.

41. The system of claim 40, wherein the expression vector is a viral vector selected from the group consisting of a lentiviral vector, an adenoviral vector, an adeno-associated viral vector, and a vaccina vector.

42. The system of any one of claims 1-41, wherein the preparation of cells is a preparation of are mammalian cells.

43. The system of claim 42, wherein the preparation of cells is a preparation of human cells.

44. The system of claim 43, wherein the preparation of cells is a preparation of pluripotent cells.

45. The system of claim 44, wherein the pluripotent cells are induced pluripotent stem cells.

46. The system of claim 44, wherein the pluripotent cells are embryonic stem cells.

47. The system of claim 43, wherein preparation of cells is a preparation of progenitor cells.

48. The system of claim 47, wherein the progenitor cells are glial progenitor cells.

49. The system of claim 47, wherein the progenitor cells are oligodendrocyte-biased progenitor cells.

50. The system of claim 47, wherein the progenitor cells are astrocyte-biased progenitor cells.

51. The system of claim 47, wherein the progenitor cells are neuronal progenitor cell.

52. The system of claim 43, wherein cells of the preparation are differentiated cells.

53. The system of claim 52, wherein the differentiated cells are neurons, oligodendrocytes, or astrocytes.

54. An in vivo method of tracking a preparation of cells implanted in a subject, said method comprising:
providing the system according to any one of claims 1-53;
implanting the preparation of cells into the subject;
administering the one or more radiolabeled binding molecules; and
detecting the one or more radiolabeled molecules when bound to its cognate cell surface binding molecule expressed by the implanted cells of the preparation, thereby tracking the cells of the implanted preparation in the subject.

55. The method of claim 54, wherein said detecting is carried out using positron emission tomography (PET) or single-photon emission computed tomography (SPECT).

56. The method of claim 54, wherein the radiolabeled molecule is a molecule that passes the blood-brain barrier.

57. The method of claim 54, wherein the subject is human.

58. The method of claim 54, wherein the preparation of cells is autologous to the subject.

59. The method of claim 54, wherein the preparation of cells is allogeneic to the subject.

60. The method of claim 54, wherein the preparation of cells is a preparation of glial progenitor cells.

61. The method of claim 54, wherein the preparation of cells and/or one or more differentiated cells thereof express a signal incompetent binding molecule selected from the group consisting of a signal incompetent dopamine receptor (DRD2), a signal incompetent serotonin receptor 4 (HTR4), a signal incompetent serotonin receptor 2 (HTR2A), a signal incompetent serotonin receptor 1B (HTR1B), a signal incompetent dopamine transporter (SLC6A3), and a signal incompetent serotonin transporter (SLC6A4).

62. The method of any one of claims 54-61, wherein the radiolabeled binding molecule is labeled with ¹²³I, ^{99m}Tc, ¹¹C, or ¹⁸F.

63. The method of any one of claim 54-62, wherein cells of the preparation or one or more differentiated cells thereof express a signal incompetent dopamine receptor, and the radiolabeled molecule is a dopamine agonist or antagonist labeled with ¹¹C or ¹⁸F.

64. The method of claim 63, wherein the radiolabeled molecule is selected from the group consisting of [¹¹C]-racloprid, 3-N-(2-[¹⁸F]-fluoroethyl)-spiperone, [¹¹C]-SCH23990, and [¹⁸F]-fallypride.

65. The method of any one of claim 54-62, wherein cells of the preparation or one or more differentiated cells thereof express a signal incompetent serotonin receptor, and the radiolabeled molecule is a selective serotonin agonist or antagonist labeled with ¹²³I, ^{99m}Tc ¹¹C, or ¹⁸F.

66. The method of claim 65, wherein the radiolabeled molecule is selected from the group consisting of [¹¹C]AZ10419369 (serotonin receptor 1B), [¹¹C]P943 (serotonin receptor 1B), [¹⁸F]Altanserin (serotonin receptor 2), [¹¹C](R)-M100907 (serotonin receptor 2), [¹¹C]CIMBI-36 (serotonin receptor 2), [¹¹C]SB207145 (serotonin receptor 4).

67. A preparation of cells, wherein said cells of the preparation are stably transduced with one or more recombinant genetic constructs, each recombinant genetic construct comprising:
a regulatory sequence driving cell-type specific gene expression; and
a nucleotide sequence encoding a cell surface binding molecule, wherein said nucleotide sequence is positioned 3′ to the regulatory sequence driving cell-type specific gene expression, and wherein the cell surface binding molecule is not endogenously expressed by cells of said preparation.

68. A preparation of cells, wherein said cells of the preparation are genetically modified with one or more recombinant genetic constructs, each construct comprising:
a first nucleotide sequence of a gene expressed in a target cell-specific manner;
a cell surface binding molecule encoding nucleotide sequence, wherein
said nucleotide sequence is positioned 3′ to the first nucleotide sequence and wherein the cell surface binding molecule is not endogenously expressed by cells of said preparation; and
a second nucleotide sequence from the same gene as the first nucleotide sequence expressed in the target cell-specific manner, said second nucleotide sequence located 3′ to the cell surface binding molecule encoding nucleotide sequence.


SEQUENCE LISTING (19 sequences)

SEQ ID NO: 1 - protein, 9 aa, Artificial Sequence
  HA Tag
YPYDVPDYA

SEQ ID NO: 2 - DNA, 27 bp, Artificial Sequence
  HA Tag
tacccatacgatgttccagattacgct

SEQ ID NO: 3 - DNA, 27 bp, Artificial Sequence
  HA Tag
tatccatatgatgttccagattatgct

SEQ ID NO: 4 - protein, 443 aa, Artificial Sequence
  dopamine receptor 2
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 5 - protein, 388 aa, Artificial Sequence
  serotonin receptor 4
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLRDAVECGGQWESQCHPPATSPLVAAQPSDT

SEQ ID NO: 6 - protein, 471 aa, Artificial Sequence
  serotonin receptor 2a
MDILCEENTSLSSTTNSLMQLNDDTRLYSNDFNSGEANTSDAFNWTVDSENRTNLSCEGCLSPSCLSLLHLQEKNWSALLTAVVIILTIAGNILVIMAVSLEKKLQNATNYFLMSLAIADMLLGFLVMPVSMLTILYGYRWPLPSKLCAVWIYLDVLFSTASIMHLCAISLDRYVAIQNPIHHSRFNSRTKAFLKIIAVWTISVGISMPIPVFGLQDDSKVFKEGSCLLADDNFVLIGSFVSFFIPLTIMVITYFLTIKSLQKEATLCVSDLGTRAKLASFSFLPQSSLSSEKLFQRSIHREPGSYTGRRTMQSISNEQKACKVLGIVFFLFVVMWCPFFITNIMAVICKESCNEDVIGALLNVFVWIGYLSSAVNPLVYTLFNKTYRSAFSRYIQCQYKENKKPLQLILVNTIPALAYKSSQLQMGQKKNSKQDAKTTDNDCSMVALGKQHSEEASKDNSDGVNEKVSCV

SEQ ID NO: 7 - protein, 390 aa, Artificial Sequence
  serotonin receptor 1b
MEEPGAQCAPPPPAGSETWVPQANLSSAPSQNCSAKDYIYQDSISLPWKVLLVMLLALITLATTLSNAFVIATVYRTRKLHTPANYLIASLAVTDLLVSILVMPISTMYTVTGRWTLGQVVCDFWLSSDITCCTASILHLCVIALDRYWAITDAVEYSAKRTPKRAAVMIALVWVFSISISLPPFFWRQAKAEEEVSECVVNTDHILYTVYSTVGAFYFPTLLLIALYGRIYVEARSRILKQTPNRTGKRLTRAQLITDSPGSTSSVTSINSRVPDVPSESGSPVYVNQVKVRVSDALLEKKKLMAARERKATKTLGIILGAFIVCWLPFFIISLVMPICKDACWFHLAIFDFFTWLGYLNSLINPIIYTMSNEDFKQAFHKLIRFKCTS

SEQ ID NO: 8 - protein, 620 aa, Artificial Sequence
  dopamine transporter
MSKSKCSVGLMSSVVAPAKEPNAVGPKEVELILVKEQNGVQLTSSTLTNPRQSPVEAQDRETWGKKIDFLLSVIGFAVDLANVWRFPYLCYKNGGGAFLVPYLLFMVIAGMPLFYMELALGQFNREGAAGVWKICPILKGVGFTVILISLYVGFFYNVIIAWALHYLFSSFTTELPWIHCNNSWNSPNCSDAHPGDSSGDSSGLNDTFGTTPAAEYFERGVLHLHQSHGIDDLGPPRWQLTACLVLVIVLLYFSLWKGVKTSGKVVWITATMPYVVLTALLLRGVTLPGAIDGIRAYLSVDFYRLCEASVWIDAATQVCFSLGVGFGVLIAFSSYNKFTNNCYRDAIVTTSINSLTSFSSGFVVFSFLGYMAQKHSVPIGDVAKDGPGLIFIIYPEAIATLPLSSAWAVVFFIMLLTLGIDSAMGGMESVITGLIDEFQLLHRHRELFTLFIVLATFLLSLFCVTNGGIYVFTLLDHFAAGTSILFGVLIEAIGVAWFYGVGQFSDDIQQMTGQRPSLYWRLCWKLVSPCFLLFVVVVSIVTFRPPHYGAYIFPDWANALGWVIATSSMAMVPIYAAYKFCSLPGSFREKLAYAIAPEKDRELVDRGEVRQFTLRHWLKV

SEQ ID NO: 9 - protein, 630 aa, Artificial Sequence
  serotonin transporter
METTPLNSQKQLSACEDGEDCQENGVLQKVVPTPGDKVESGQISNGYSAVPSPGAGDDTRHSIPATTTTLVAELHQGERETWGKKVDFLLSVIGYAVDLGNVWRFPYICYQNGGGAFLLPYTIMAIFGGIPLFYMELALGQYHRNGCISIWRKICPIFKGIGYAICIIAFYIASYYNTIMAWALYYLISSFTDQLPWTSCKNSWNTGNCTNYFSEDNITWTLHSTSPAEEFYTRHVLQIHRSKGLQDLGGISWQLALCIMLIFTVIYFSIWKGVKTSGKVVWVTATFPYIILSVLLVRGATLPGAWRGVLFYLKPNWQKLLETGVWIDAAAQIFFSLGPGFGVLLAFASYNKFNNNCYQDALVTSVVNCMTSFVSGFVIFTVLGYMAEMRNEDVSEVAKDAGPSLLFITYAEAIANMPASTFFAIIFFLMLITLGLDSTFAGLEGVITAVLDEFPHVWAKRRERFVLAVVITCFFGSLVTLTFGGAYVVKLLEEYATGPAVLTVALIEAVAVSWFYGITQFCRDVKEMLGFSPGWFWRICWVAISPLFLLFIICSFLMSPPQLRLFQYNYPYWSIILGYCIGTSSFICIPTYIAYRLIITPGTFKERIIKSITPETPTEIPCGDIRLNAV

SEQ ID NO: 10 - DNA, 600 bp, Artificial Sequence
  Puromycin Resistance
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagggcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 11 - DNA, 816 bp, Artificial Sequence
  Neomycin Resistance
atgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 12 - DNA, 1026 bp, Artificial Sequence
  Hygromycin B
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctcgagccggtcgcggaggcgatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcatgcggatttcggctccaacaatgtcctgacggacaatggccgcataacagcggtcattgactggagcgaggcgatgttcggggattcccaatacgaggtcgccaacatcctcttctggaggccgtggttggcttgtatggagcagcagacgcgctacttcgagcggaggcatccggagcttgcaggatcgccgcgcctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 13 - DNA, 66 bp, Artificial Sequence
  Porcine teschovirus-1 2A (P2A)
ggaagcggagctactaacttcagcctgctgaagcaggctggagacgtggaggagaaccctggacct

SEQ ID NO: 14 - DNA, 66 bp, Artificial Sequence
  Porcine teschovirus-1 2A (P2A), codon optimized
ggttccggagccacgaacttctctctgttaaagcaagcaggagacgtggaagaaaaccccggtccc

SEQ ID NO: 15 - DNA, 75 bp, Artificial Sequence
  Foot and mouth disease virus 2A (F2A)
ggaagcggagtgaaacagactttgaattttgaccttctcaagttggcgggagacgtggagtccaaccctggacct

SEQ ID NO: 16 - DNA, 54 bp, Artificial Sequence
  Thosea asigna virus 2A (T2A)
gagggcagaggaagtcttctaacatgcggtgacgtggaggagaatcccggccct

SEQ ID NO: 17 - DNA, 69 bp, Artificial Sequence
  Equine rhinitis A virus 2A (E2A)
ggaagcggacagtgtactaattatgctctcttgaaattggctggagatgttgagagcaaccctggacct

SEQ ID NO: 18 - DNA, 66 bp, Artificial Sequence
  Cytoplasmic polyhedrosis virus (BmCPV 2A)
gacgtttttcgctctaattatgacctactaaagttgtgcggtgatatcgagtctaatcctggacct

SEQ ID NO: 19 - DNA, 66 bp, Artificial Sequence
  Flacherie virus (BmIFV 2A)
actctgacgagggcgaagattgaggatgaattgattcgtgcaggaattgaatcaaatcctggacct